(19) United States
(12) Patent Application Publication
(10) Pub. No.: US 2017/0096426 A1
(43) Pub. Date: Apr. 6, 2017

(54) Novel Tricyclic Modulators of Cannabinoid Receptors

(71) Applicant: The University of Montana, Missoula, MT (US)

(72) Inventors: Philippe Diaz, Missoula, MT (US); Fanny Diaz, Missoula, MT (US); Ravil R. Petrov, Missoula, MT (US)

(21) Appl. No.: 15/288,592

(22) Filed: Oct. 7, 2016

Related U.S. Application Data
(63) Continuation of application No. 13/154,234, filed on Jun. 6, 2011, now abandoned.
(60) Provisional application No. 61/351,429, filed on Jun. 4, 2010.

(51) Int. Cl. C07D 471/04; G01N 33/94; C07D 405/06; C07D 401/06; C07D 209/88; C07D 209/82

(52) U.S. Cl. CPC C07D 471/04; C07D 209/88; C07D 209/82; C07D 405/06; C07D 401/06; G01N 33/948

(57) ABSTRACT

The compounds of the invention are modulators of cannabinoid receptors CB1 or CB2. The compounds can be used for the prevention or treatment of, e.g., pain, cancer, skin diseases, weight-associated disorders, chemical addictions, psychiatric disorders, neurodegenerative disorders, bone diseases, and inflammatory diseases. The compounds of the invention can further be used to study these diseases and disorders, as well as cannabinoid receptor biology, by coupling the compounds to, e.g., imaging agents.

### CROSS-REFERENCE TO RELATED APPLICATIONS

This application is a continuation of U.S. patent application Ser. No. 13/154,234, filed Jun. 6, 2011, now abandoned, which claims benefit of U.S. Provisional Application No. 61/351,429, filed Jun. 4, 2010, the disclosures of which are hereby incorporated by reference in their entirety including all figures, tables and drawings.

### STATEMENT REGARDING FEDERALLY SPONSORED RESEARCH OR DEVELOPMENT

This invention was made with government support under Grant No. P30 NS055022 awarded by the National Institutes of Health. The government has certain fights in the invention.

### REFERENCE TO SEQUENCE LISTING, A TABLE, OR A COMPUTER PROGRAM LISTING COMPACT DISC APPENDIX

Not applicable.

### BACKGROUND OF THE INVENTION

Historically, cannabinoid preparations, derived from the hemp Cannabis saliva L., have been used for medicinal and recreational purposes for many centuries. The main active ingredient in cannabis, tetrahydrocannabinol (Δ 9-THC), was identified in 1964 (Gaoni et al., J. Am. Chem. Soc. 86:1646-7 (1964)). Two cannabinoid receptors belonging to the G-protein-coupled receptor family have been identified, CB1 and CB2, along with seven endogenous lipid ligands and the enzymes involved in their syntheses and metabolism (Matsuda et al., Nature 346:561-4 (1990)).

Neuropathic pain is caused by a lesion in the central (brain and spinal cord) or peripheral nervous system. It is not a single disease entity and may result from a wide range of heterogeneous conditions that differ in etiology. It is triggered by conditions such as diabetic neuropathy, MDS-related neuropathy, post-herpetic neuralgia, degenerative spinal disease, chemotherapy, radiotherapy, sympathetic dystrophies, post-amputation stump (phantom limb pain), trigeminal neuralgia, and multiple sclerosis (MS). Allodynia (touch-evoked pain) and hyperalgesia are clinically perplexing characteristics of neuropathic pain.

The prevalence of neuropathic pain is estimated to be about 8% in the general population worldwide (Torrance et al., J. Pain 7:281-289 (2006)). In the U.S., the annual healthcare cost attributable to neuropathic pain is almost $40 billion (Turk, Clin. J. Pain 18:355-65 (2002)). Currently, there is no effective or satisfactory treatment for neuropathic pain (Warms et al., Clin. J. Pain 18:154-63 (2002)).

Two cannabinoid (CB) receptors (CB1 and CB2) have been characterized and cloned (Matsuda et al., Nature (1990)); Munro et al., Nature 365:61-5 (1993)). CB1 is expressed in the central nervous system as well as in the peripheral nervous system. The CB1 receptor is found predominantly in the brain, with highest densities in the hippocampus, cerebellum, and striatum (Ameri, Prog. Neurobiol. 58:315-348 (1999)). Impairment of cognitive functions induced by Δ9-THC is mediated by CB1 receptors in the hippocampus (Herkenham et al., Proc. Natl. Acad. Sci. USA 87:1932-1936 (1990)). Despite the promising effects of CB1 agonists on pain relief, CNS side effects such as catalepsy or motor impairment have compromised their pharmaceutical development.

CB2 receptors are expressed predominately in immune tissues; including the spleen, tonsils, monocytes, and B and T lymphocytes, although CB2 receptors and their gene transcripts are widely distributed in the CNS (Munro et al., Nature (1993); Facci et al., Proc. Natl. Acad. Sci. USA 92:3376-80 (1995); and Onaivi et al., Ann. NY Acad. Sci. 1074:514-536 (2006)).

The multifocal expression of CB2 immunoreactivity in the brain suggests that CB2 receptors play a role in the brain and may be involved in depression and substance abuse. See e.g., Onaivi et al., NY Acad. Sci. (2006); Berghuis et al., Science 316:1212-1216 (2007); Kaki et al., Nat. Clin. Pract. Urol. 2:492-501 (2005); Kathuria et al., Nat. Med. 9: 76-81 (2003); Baker et al., Nature 404:84-87 (2000)). Furthermore; the endocannahinoid system has been implicated in allergic contact dermatitis (Karsak et al., Science 316:1494-7 (2007).

In addition, studies provide support for the role of cannabinoid system in several physiological functions including food consumption and body weight, in which CB1 receptor activation leads to increased food consumption and weight gain (Fride, Prostaglandins Leukot. Essent. Fatty Acids 66:221-33 (2002)). Subsequently, CB1 receptor blockade reduces food consumption and leads to weight loss (Van Gaal et al., The Lancet 365:1389-1397 (2005)).

Modulators of CB1/CB2 receptors have been used in different clinical or preclinical studies (Steffens et al., Nature 434:782-786 (2005)). For example, CB1 agonists have been used for treatment of nausea, Tourette's Syndrome, Parkinson's Disease, glaucoma, cancer, diarrhea, and stroke (Guzman, Nature Reviews Cancer 3:745-755 (2003)). Further, CB2 agonists have been used for treatment pain, gliomas, lymphomas, and inflammation (Maresz et al., Nat. Med. 13:492-497 (2007)).

Unlike CB1 agonists, CB2 ligands are devoid of psychoactivity. Up-regulation of CB2-receptor mRNA and proteins in the dorsal root ganglia and spinal cord is also found in animals after spinal nerve ligation; sciatic nerve injury, or saphenous nerve ligation (Beltramo et al., Eur. J. Neurosci. 23:1530-8 (2006); Wotherspoon et al., Neurosci. 135:235-45 (2005); Zhang et al., Eur. J. Neurosci. 17:2750-4 (2003); Walczak et al., Neurosci. 132:1093-102 (2005); Walczak et al., J. Neurosci. Res. 83:1310-22 (2006)). CB2 receptor activation potentiates obesity-associated inflammation, insulin resistance, and hepatic steatosis (Deveaux et al., PLoS ONE 4(6):e5844 (2009)).

CB1 antagonists have been used for treatment obesity and addiction (Crowley et al., Nature Reviews Drug Discovery 1:276-286 (2002); Trang et al., Neurosci. 146:1275-1288 (2007); Teixeira-Clerc et al., Nat. Med. 12:671-676 (2006)). For example, the CB1 antagonist SR141716A reduces food intake in mice (Di Marzo et al., Nature 410:822-5 (2001)). CB1 cannabinoid antagonists can also be used to treat drug addiction (Maldonado et al., Trends Neurosci. 2006; 29:225-32 (2006)). Cannabinoids attenuate deep tissue hyperalgesia produced by both cancer and inflammatory conditions (Kehl et al., Pain 103:175-86 (2003)). Cannabinoids also can be used for the treatment osteoporosis and other bone diseases (Idris et al., Nat. Med. 11:774-9 (2005)). Cannabinoids are able to reduce intraocular pressure. CB1 has also been shown to be involved in ectopic pregnancy in mice (Wang et al., Nat. Med. 10:1074-1080 (2004)).

Certain published data demonstrate that human keratinocytes partake in the peripheral endocannabinoid system. CB1 receptors have been implicated in epidermal differentiation and skin development (Maccarrone et al., J. Biol. Chem. 2003, 278:33896-903 (2003)). Hence, cannabinoid modulator can be useful in the treatment of skin diseases.

Recently it has been shown that cannabinoids inhibit keratinocyte proliferation, and therefore support a potential role for cannabinoids in the treatment of psoriasis (Wilkinson et al., J. Dermatol. Sci. 45:87-92 (2007)). Cannabinoid receptors are also targets for the treatment of melanoma (Blazquez et al., Faseb J. 20:2633-5 (2006)). The anti-pruritic activity of CB2 modulators was studied in NC mice with chronic dermatitis, a model of atopic dermatitis. Hence, cannabinoid CB2 receptor modulators may also be useful for the treatment of pruritus (Maekawa et al., Eur. J. Pharmacol. 542 179-183 (2006)).

Many diseases and disorders are poorly understood and ineffectively treated. For example, drugs used for the treatment of neuropathic pain or obesity-associated disorders are not effective. Current treatments are aimed at alternate biological targets to reduce the pain signal or as analgesia. Treatment of obesity-associated disorders include attempts to reduce obesity-associated inflammation. These ineffective or analgesic treatments often result in dependency on expensive drugs with subsequent tolerance build-up while not addressing the underlying medical problems. A need remains for a means by which diseases, disorders, conditions, or symptoms that involve cannabinoid receptor signaling can be studied and effectively treated.

All patents, patent applications, provisional patent applications, and publications referred to or cited herein, are incorporated by reference in their entirety to the extent they are not inconsistent with the explicit teachings of the specification.

### BRIEF SUMMARY OF THE INVENTION

In one aspect of the invention, tricyclic compounds that can bind or modulate cannabinoid receptors, such as cannabinoid receptor 1 (CB1) or 2 (CB2), are presented and defined by the structural Formula I:

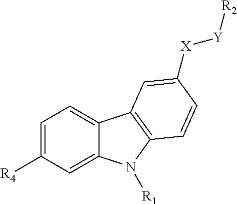

or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkyl carbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, a hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, cycloalkyl, aralkyl, alkenyl, alkynyl, a halogen, alkoxy, and hydroxyl.

In one embodiment, the compound is N-[2-(4-chlorophenyl)ethyl]-9-pentyl-9H-carbazole-3-carboxamide; 9-(cyclohexylmethyl)-2-methoxy-6-[(piperidin-1-yl)carbonyl]-9H-carbazole; 2-methoxy-9-pentyl-6-[(piperidin-1-yl)carbothioyl]-9H-carbazole; 9-pentyl-3-[(piperidin-1-yl)carbothioyl]-9H-carbazole; 9-pentyl-6-[(piperidin-1-yl)carbonyl]-9H-carbazol-2-ol; 2-[(ethylsulfanyl)methoxy]-9-pentyl-6-[(piperidin-1-yl)carbonyl]-9H-carbazole; 2-methoxy-6-[(morpholin-4-yl)carbonyl]-9-pentyl-9H-carbazole; 3-[(morpholin-4-yl)carbonyl]-9-pentyl-9H-carbazole; 2-methoxy-6-[(4-methylpiperazin-1-yl)carbonyl]-9-pentyl-9H-carbazole; 3-[(4-methylpiperazin-1-yl)carbonyl]-9-pentyl-9H-carbazole; 7-methoxy-9-pentyl-N-(piperidin-1-yl)-9H-carbazole-3-carboxamide; 9-pentyl-N-(piperidin-1-yl)-9H-carbazole-3-carboxamide; N,N-diethyl-7-methoxy-9-pentyl-9H-carbazole-3-carboxamide; N,N-diethyl-9-pentyl-9H-carbazole-3-carboxamide; N-(adamantan-1-yl)-7-methoxy-9-pentyl-9H-carbazole-3-carboxamide; N-(adamantan-1-yl)-9-pentyl-9H-carbazole-3-carboxamide; 2-(methylsulfanyl)-9-pentyl-6-[(piperidin-1-yl)carbonyl]-9H-carbazole; 2-methanesulfonyl-9-pentyl-6-[(piperidin-1-yl)carbonyl]-9H-carbazole; N-[2-(4-chlorophenyl)ethyl]-7-methoxy-9-pentyl-9H-carbazole-3-carboxamide; 4-[(7-methoxy-9-pentyl-9H-carbazol-3-yl)carbonyl]-1,1-dimethylpiperazin-1-ium iodide; 1,1-dimethyl-4-[(9-pentyl-9H-carbazol-3-yl)carbonyl]piperazin-1-ium iodide; 4-[(9-pentyl-9H-carbazol-3-yl)carbonyl]-1λ⁶,4-thiomorpholine-1,1-dione; dimethyl(3-{3-[(piperidin-1-yl)carbonyl]-9H-carbazol-9-yl}propyl)amine; 9-(3-methoxypropyl)-3-[(piperidin-1-yl)carbonyl]-9H-carbazole; methyl 4-{3-[(piperidin-1-yl)carbonyl]-9H-carbazol-9-yl}butanoate; 3-benzoyl-9-pentyl-9H-carbazole; 9-(oxan-4-ylmethyl)-3-[(piperidin-1-yl)carbonyl]-9H-carbazole; 3-[(piperidin-1-yl)carbonyl]-9-(pyridin-4-ylmethyl)-9H-carbazole; 3-[(piperidin-1-yl)carbonyl]-9-(pyridin-3-ylmethyl)-9H-carbazole; 9-ethyl-3-[(4-methylnaphthalen-1-yl)carbonyl]-9H-carbazole; 2-methoxy-9-pentyl-6-[(piperidin-1-yl)carbonyl]-9H-carbazole; 9-pentyl-3-[(piperidin-1-yl)carbonyl]-9H-carbazole; 3-[(piperidin-1-yl)carbonyl]-9-(pyridin-2-ylmethyl)-9H-carbazole; methyl 9-(cyclohexylmethyl)-7-methoxy-9H-carbazole-3-carboxylate; ethyl 9-pentyl-9H-pyrido[3,4-b]indole-3-carboxylate; N-(2,2-dimethylpropyl)-9-pentyl-9H-pyrido[3,4-b]indole-3-carboxamide; 1-({9-pentyl-9H-pyrido[3,4-b]indol-3-yl}carbonyl)piperidine; 9-pentyl-N-(piperidin-1-yl)-9H-pyrido[3,4-b]indole-3-carboxamide, 2-methoxy-9-pentyl-6-(piperidin-1-ylmethyl)-9H-carbazole; methyl 9-[3-(dimethylamino)propyl]-7-methoxy-9H-carbazole-3-carboxylate; or 2-(dimethylamino)ethyl 9-pentyl-9H-carbazole-3-carboxylate. In another embodiment, the compound is coupled to an imaging agent.

In a second aspect of the invention, tricyclic compounds that can bind or modulate cannabinoid receptors are defined by structural Formula II:

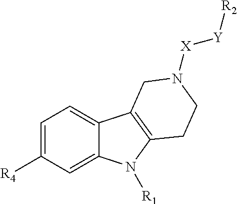

or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, alkyl, cycloalkyl, aralkyl, alkenyl, alkynyl, a halogen, alkoxy, and hydroxyl.
   In one embodiment, the compound is 2-benzoyl-7-methoxy-5-pentyl-1H,2H,3H,4H,5H-pyrido[4,3-b]indole, 5-{7-methoxy-5-pentyl-1H,2H,3H,4H,5H-pyrido[4,3-b]indole-2-sulfonyl}-N,N-dimethylnaphthalen-1-amine; and 5-ethyl-7-methoxy-2-[(4-methylnaphthalen-1-yl)carbonyl]-1H,2H,3H,4H,5H-pyrido[4,3-b]indole. In another embodiment, the compound is coupled to an imaging agent.

In a third aspect of the invention, tricyclic compounds that can bind or modulate cannabinoid receptors are defined by structural Formula III:

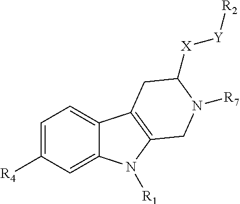

or a salt, ester or prodrug thereof, wherein
X is selected from the group consisting of C═O, C═S, S═O and SO2;
Y is selected from the group consisting of O, N—R³, and R³—C—R⁸,
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted;
R⁷ is selected from the group consisting of, polyether radical, alkylcarbonyl, heteroaryl, heterocycloalkyl, aryl, alkyl, cycloalkyl, aralkyl, alkenyl, and alkynyl; and
R⁸ is selected from the group consisting of hydrogen, aryl, alkyl, cycloalkyl, aralkyl, alkenyl, alkynyl, a halogen, alkoxy, and hydroxyl.
   In one embodiment, the compound is 1-{[(3R)-9-pentyl-1H,2H,3H,4H,9H-pyrido[3,4-b]indol-3-yl]carbonyl}piperidine hydrochloride; 1-{[(3S)-9-pentyl-1H,2H,3H,4H,9H-pyrido[3,4-b]indol-3-yl]carbonyl}piperidine hydrochloride; (3R)-2,2-dimethyl-9-pentyl-3-[(piperidin-1-yl)carbonyl]-1H,2H,3H,4H,9H-pyrido[3,4-b]indol-2-ium iodide; (3S)-2,2-dimethyl-9-pentyl-3-[(piperidin-1-yl)carbonyl]-1H,2H,3H,4H,9H-pyrido[3,4-b]indol-2-ium iodide; or 1-{1 [(3R)-2,9-dipentyl-1H,2H,3H,4H,9H-pyrido[3,4-b]indol-3-yl]carbonyl}piperidine hydrochloride. In another embodiment, the compound is coupled to an imaging agent.

In a fourth aspect of the invention, a patient (e.g., a human) suffering from a symptom, disease, or condition can be treated by administering to the patient a therapeutically effective amount of a tricyclic compound defined by structural formulae I-III. In one embodiment, the symptom, disease, or condition treated is pain, cancer, a skin disease, a weight-associated disorder, chemical addiction, a psychiatric disorder, a neurodegenerative disorder, a bone disease, or an inflammatory disease. In another embodiment, the symptom, disease, or condition treated is neuropathic pain. In a further embodiment, the symptom, disease, or condition treated is a skin disease such as psoriasis, contact dermatitis, atopic dermatitis, eczema, melanoma, itch, or pruritus. In yet a further embodiment, the symptom, disease, or condition treated is a weight-associated disorder such as obesity, anorexia nervosa, bulimia nervosa, exercise bulimia, binge eating disorder, or weight loss.

In a fifth aspect of the invention, a tricyclic compound defined by any one of structural formulae I-III can be used to detect a cannabinoid receptor. In one embodiment, the compound is used to detect cannabinoid receptor 1 (CB1). In another embodiment, the compound is used to detect cannabinoid receptor 2 (CB2).

In a sixth aspect of the invention, a tricyclic compound defined by any one of structural formulae I-III can be used to modulate a cannabinoid receptor. In one embodiment, the compound is used to detect cannabinoid receptor 1 (CB1). In another embodiment, the compound is used to detect cannabinoid receptor 2 (CB2).

As used herein, the terms below have the meanings indicated.

The term “acyl,” as used herein, alone or in combination, refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(O)CH₃ group.

An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds optionally substituted and containing from 2 to 20, preferably 2 to 6, carbon atoms. Alkenylene refers to a carbon-carbon double bond system attached at two or more positions such as ethenylene [(—CH═CH—),(—C::C—)]. Examples of alkenyl radicals include ethenyl, propenyl, 2-methylpropenyl, 1,4-butadienyl and the like.

The term “alkoxy,” as used herein, alone or in combination, refers to an alkyl ether radical, optionally substituted wherein the term alkyl is as defined below. Examples of alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical optionally substituted containing from 1 to 20 and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like.

The term “alkylamino,” as used herein, alone or in combination, refers to an alkyl group optionally substituted attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The term “alkylthio,” as used herein, alone or in combination, refers to an alkyl thioether (R—S—) radical wherein the term alkyl is as defined above and wherein the sulfur may be singly or doubly oxidized. Examples of alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl” as used herein, alone or in combination, refer to an amino group as described below attached to the parent molecular moiety through a carbonyl group, or vice versa.

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused optionally substituted with at least one halogen, an alkyl containing from 1 to 3 carbon atoms, an alkoxyl, an aryl radical, a nitro function, a polyether radical, a heteroaryl radical, a benzoyl radical, an alkyl ester group, a carboxylic acid, a hydroxyl optionally protected with an acetyl or benzoyl group, or an amino function optionally protected with an acetyl or benzoyl group or optionally substituted with at least one alkyl containing from 1 to 12 carbon atoms.

The term “arylalkyl” or “aralkyl,” as used herein, alone or in combination, refers to an aryl group attached to the parent molecular moiety through an alkyl group.

The term aryloxy as used herein, alone or in combination, refers to an aryl group attached to the parent molecular moiety through an oxygen atom.

The term “polyether radical” means a polyether radical containing from 2 to 6 carbon atoms interrupted with at least one oxygen atom, such as methoxymethyl, ethoxymethyl or methoxyethoxymethyl radicals or methoxyethyl.

The terms “benzo” and “benz,” as used herein, alone or in combination, refer to the divalent radical C₆H₄═ derived from benzene. Examples include benzothiophene and benzitnidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo-fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxygen atom bridging two moieties linked at carbon atoms.

The term “halo,” or “halogen,” as used herein, alone or in combination, refers to fluorine, chlorine, bromine, or iodine.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocyclyl,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring; and most preferably 5 to 6 ring members in each ring. “Heterocycloalkyl” and “heterocyclyl” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocyclyl groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, morpholinyl, piperazinyl, pyrrolidinyl, tetrahydropyridinyl, piperidinyl, thiomorpholinyl, and the like. The heterocyclyl groups may be optionally substituted unless specifically prohibited.

The term “hydroxy,” as used herein, alone or in combination, refers to —OH.

The phrase “in the main chain” refers to the longest contiguous or adjacent chain of carbon atoms starting at the point of attachment of a group to the compounds of this invention.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “nitro,” as used herein, alone or in combination, refers to —NO₂.

The term “perhaloalkyl” as used herein, alone or in combination, refers to an alkyl group where all of the hydrogen atoms are replaced by halogen atoms.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl; carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, aryl sulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₇CF₃), Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Optical isomers are compounds with the same molecular formula but differ in the way they rotate plane polarized light. There are two types of optical isomers. The first type of optical isomers are compounds that are mirror images of one another but cannot be superimposed on each other. These isomers are called “enantiomers.” The second type of optical isomers are molecules that are not mirror images but each molecule rotates plane polarized light and are considered optically-active. Such molecules are called “diastereoisomers.” Diasteroisomers differ not only in the way they rotate plane polarized light, but also their physical properties. The term “optical isomer” comprises more particularly the enantiomers and the diastereoisomers, in pure form or in the form of a mixture.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “bone disease” refers to any disease, disorder, or condition relating to the bone, including, e.g., osteoporosis, osteoarthritis, and osteomyelitis.

The terms “cancer” refers to or describes the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“Cannabinoid receptor modulator” is used herein to refer to a compound that exhibits an EC₅₀ or IC₅₀ with respect to a cannabinoid receptor activity of no more than about 50 μM and more typically not more than about 10 μM, as measured in the cannabinoid receptor assay described herein. “EC₅₀” is that concentration of modulator which activates the activity of a cannabinoid receptor to half-maximal level. “IC₅₀” is that concentration of modulator which reduces the activity of a cannabinoid receptor to half-maximal level.

The term “imaging agent” as used herein refers to any moiety useful for the detection, tracing, or visualization of a compound of the invention when coupled thereto.

Imaging agents include, e.g., an enzyme, a fluorescent label (e.g., fluorescein), a luminescent label, a bioluminescent label, a magnetic label, a metallic particle (e.g., a gold particle), a nanoparticle, an antibody or fragment thereof (e.g., a Fab, Fab′, or F(ab′)₂ molecule), and biotin. An imaging agent can be coupled to a compound of the invention by, for example, a covalent bond, ionic bond, van der Waals interaction or a hydrophobic bond. An imaging agent of the invention can be a radiolabel coupled to a compound of the invention, or a radioisotope incorporated into the chemical structure of a compound of the invention. Methods of detecting such imaging agents are well known to those having skill in the art.

The term. “inflammatory disease” as used herein refers to any disease, disorder, condition, or symptom characterized by an inflammatory process, including, e.g., autoimmunity (e.g., inflammatory bowel disease, rheumatoid arthritis, and multiple sclerosis), cancer, atopy (e.g., asthma), atherosclerosis, and ischemic heart disease.

The term “itch” is used herein in the broadest sense and refers to all types of itching and stinging sensations localized and generalized, acute intermittent and persistent. The itch may be idiopathic, allergic, metabolic, infectious, drug-induced, due to liver, kidney disease, or cancer. “Pruritus” is severe itching.

The term “modulator” described herein reflects any chemical compound that will act as full agonist, partial agonist, inverse agonist or as an antagonist on a cannabinoid receptor. Compounds described herein have been discovered to exhibit modulatory activity against cannabinoid receptors and exhibit an EC₅₀ of IC₅₀ with respect to a cannabinoid receptor of no more than about 10 μM, more preferably, no more than about 5 μM, even more preferably not more than about 1 μM, and most preferably, not more than about 200 nM, as measured in the assays described herein.

The term “neurodegenerative disorder” as used herein refers to any disease, disorder, condition, or symptom characterized by the structural or functional loss of neurons. Neurodegenerative disorders include, e.g., Alzheimer's Disease, Parkinson's Disease, Huntington's Disease, and amyotrophic lateral sclerosis.

The term “pain” is used herein in the broadest sense and refers to all types of pain, including acute and chronic pain, such as nociceptive pain, e.g., somatic pain and visceral pain, inflammatory pain, dysfunctional pain, idiopathic pain, neuropathic pain, e.g., centrally generated pain and peripherally generated pain, migraine, and cancer pain.

The term “nociceptive pain” is used to include all pain caused by noxious stimuli that threaten to or actually injure body tissues, including, without limitation, by a cut, bruise, bone fracture, crush injury, burn, and the like. Pain receptors for tissue injury (nociceptors) are located mostly in the skin, musculoskeletal system, and internal organs.

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associates with active inflammation that may be caused by trauma, surgery, infection and autoimmune diseases.

The term “neuropathic pain” is used herein to refer to pain originating from abnormal processing of sensory input by the peripheral or central nervous system consequent on a lesion to these systems. Neuropathic pain in a patient can be caused by, e.g., diabetic neuropathy, AIDS-related neuropathy, post-herpetic neuralgia, trigeminal neuralgia, chemotherapy, radiotherapy, multiple sclerosis, sympathetic dystrophy, allodynia, and hyperalgesia.

The term “procedural pain” refers to pain arising from a medical, dental or surgical procedure. Procedural pain can result from treatment, e.g., with chemotherapy or radiotherapy.

The term “psychiatric disorder” as used herein refers to any mental disease, disorder, condition, or symptom including, e.g., depression, dysthymia, seasonal affective disorder, postpartum depression, bipolar disorder, anxiety, schizophrenia, Tourette's Syndrome, and obsessive-compulsive-disorder.

The term “skin disease” as used herein refers to any disease, disorder, condition, or symptom of the skin, including, e.g., psoriasis, contact dermatitis, atopic dermatitis, eczema, melanoma, itch, and pruritus.

The phrase “therapeutically effective” is intended to qualify the amount of active ingredients used in the treatment of a disease or disorder. This amount will achieve the goal of reducing or eliminating the disease or disorder.

The term “therapeutically acceptable” refers to those compounds (or salts, esters, prodrugs, tautomers, zwitterionic forms, etc. thereof) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, rabbits, and rodents (e.g., rats, mice, and guinea pigs).

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology, Testa, Bernard and Wiley-VHCA, Zurich, Switzerland 2003. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bio-available by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound which is administered as an ester (the “prodrug”), but then is metabolically hydrolyzed to the carboxylic acid, the active entity. Additional examples include peptidyl derivatives of a compound.

The compounds of the invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Stahl, P. Heinrich, Pharmaceutical Salts: Properties, Selection, and Use, Wiley-VCHA, Zurich, Switzerland (2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, naphthalenesulfonate, oxalate, pamoate, pectinate, persulfate, 3-phenylproprionate, phosphonate, picrate, pivalate, propionate, pyroglutamate, succinate, sulfonate, tartrate, tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

The term “weight-associated disorder” refers to any disease, disorder, condition, or symptom associated with abnormal weight gain or loss in a patient. Weight-associated disorders include, e.g., obesity, anorexia nervosa, bulimia nervosa, exercise bulimia, binge eating disorder, and weight loss.

### BRIEF DESCRIPTION OF THE DRAWINGS

FIG. 1A-X is a table listing the chemical structure, chemical name, melting point, molecular weight (theoretical and as determined by mass spectrometer), retention time, and formula of the invention classification for specific embodiments of the compounds of the invention.

FIG. 2 is a table showing the results of radioligand competitive binding assays and the GTPγ[³⁵S] functional activity of selected compounds of the invention.

### DETAILED DESCRIPTION OF THE INVENTION

The present invention involves novel, small molecule carbazole and carboline analogs that modulate cannabinoid receptors such as CB1 and CB2, and can be used for the prevention and treatment of diseases, disorders, conditions, and symptoms in a patient (e.g., a human) including pain (e.g., neuropathic pain), cancer, skin diseases (e.g., itch, pruritus, and melanoma), obesity-associated disorders (e.g., anorexia nervosa, bulimia nervosa, and weight loss), chemical addictions (e.g., alcohol and drug addiction), psychiatric disorders (e.g., depression, bipolar disorder, and schizophrenia), neurodegenerative disorders (e.g., Alzheimer's Disease), bone diseases (e.g., osteoporosis), and inflammatory diseases (e.g., rheumatoid arthritis and multiple sclerosis). The compounds of the invention can also be used to study the biological and chemical mechanisms of these diseases, disorders, conditions, and symptoms by coupling the compound to, for example, an imaging agent (e.g., a fluorochrome or radioisotope). The compounds of the invention can also be used as research tools in the study of cannabinoid receptor biology and related processes by, for example, modulating a cannabinoid receptor.

A class of tricyclic compounds is presented and defined by the structural Formula I:

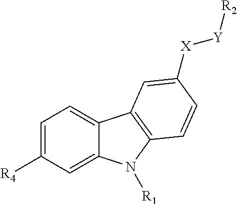

or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl; alkynyl; heteroaryl, a halogen, alkoxy, heterocycloalkyl, polyether radical, a hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, alkyl, cycloalkyl, aralkyl, alkenyl, alkynyl, a halogen, alkoxy, and hydroxyl;
   by the structural Formula II:

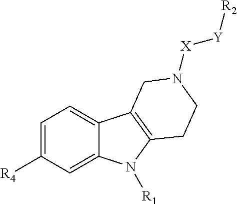

or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, alkyl, cycloalkyl, aralkyl, alkenyl, alkynyl, a halogen, alkoxy, and hydroxyl;
   and by the structural Formula III:

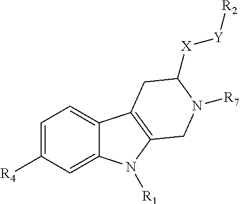

or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted;
R⁷ is selected from the group consisting of polyether radical, alkylcarbonyl, heteroaryl, heterocycloalkyl, aryl, alkyl, cycloalkyl, aralkyl, alkenyl, and alkynyl; and
R⁸ is selected from the group consisting of hydrogen, aryl, alkyl, cycloalkyl, alkenyl, alkynyl, a halogen, alkoxy, and hydroxyl.

### Methods of Prevention and Treatment

The compounds of the invention can be used to treat a patient (e.g., a human) that suffers from or is at risk of suffering from a disease, disorder, condition or symptom described herein. The compounds of the invention can be used alone or in combination with other agents and compounds in the treatment of, e.g., neuropathic pain, addiction (e.g., addiction caused by nicotine, cocaine, opioids, hashish, marijuana, alcohol dependence, and food), cancer (e.g., melanoma, lymphoma, and glioma), inflammation (e.g., autoimmune inflammation and weight-associated inflammation), cardiovascular disease, liver fibrosis, obesity, insulin resistance, hepatic steatosis, osteoporosis, and other bone diseases. Additional indications for use of the compounds disclosed herein include acne, psoriasis, allergic contact dermatitis, anxiety, spasticity and tremor, bladder dysfunctions, prevention of miscarriage, ectopic pregnancy, Tourette's Syndrome, Parkinson's Disease, stroke, glaucoma, diseases of the eye (e.g., intraocular pressure), diarrhea, and nausea. Each such treatment described above includes the step of administering to a patient in need thereof a therapeutic effective amount of the compound of the invention described herein to reduce or prevent such disease, disorder, condition, or symptom.

Besides being useful for human treatment, the compounds and formulations of the present invention are also useful for the treatment of animals, e.g., the veterinary treatment of companion animals (e.g., dogs and cats), exotic animals, farm animals (e.g., ungulates, including horses, cows, sheep, goats, and pigs), and animals used in scientific research (e.g., rodents)

Therefore, the compounds of the invention described herein may be used alone or in combination with another agent or compound in methods for treating, ameliorating or preventing a syndrome, disorder or disease in which a cannabinoid receptor is involved, including, but not limited to, ocular complaint such as glaucoma, pain, controlling appetite, regulating metabolism, diabetes, social and mood disorders, seizure-related disorders, substance abuse disorders, learning, cognition and/or memory disorders, bowel disorders, gastrointestinal disorders, respiratory disorders, locomotor activity disorders, movement disorders, immune disorders or inflammation disorders, and controlling organ contraction and muscle spasm.

The compounds of the invention presented herein may be also useful in enhancing learning, cognition and/or memory, regulating cell growth, providing neuroprotection and the like. The compounds presented herein may also be used for treating dermatological complaints associated with a keratinization disorder relating to cell differentiation and proliferation, especially for treating acne, for treating other dermatological complaints, with or without cell proliferation disorder, and especially all forms of psoriasis, for treating all dermal or epidermal proliferations, for preventing or treating cicatrization disorders, in the treatment of dermatological or general complaints with an immunological component, in the treatment of skin disorders caused by exposure to UV radiation, and also for combating sebaceous function disorders, for repairing or combating aging of the skin, for preventing or treating cicatrization disorders, or in the treatment of pigmentation disorders.

### Compound Administration and Formulation

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanol amine, piperidine, and piperazine.

A salt of a compound can be made by reacting the appropriate compound in the form of the free base with the appropriate acid. The novel compounds described herein can be prepared in a form of pharmaceutically acceptable salts that will be prepared from nontoxic inorganic or organic bases including but not limited to aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally-occurring substituted amines, cyclic amines; and basic ion exchange resins, such as argmine, betaine, caffeine, choline, ethylamine, 2-diethylaminoethano, 1,2-dimethylaminoethanol, ethanolarnine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, and tromethamine.

If the compounds of the invention are basic, salts could be prepared in a form of pharmaceutically acceptable salts that will be prepared from nontoxic inorganic or organic acids including but not limited to hydrochloric, hydrobromic, phosphoric; sulfuric, tartaric, citric, acetic, fumaric, alkylsulphonic, naphthalenesulphonic, para-toluenesulphonic, camphoric acids, benzenesulfonic, benzoic, camphorsulfonic, citric, ethanesulfonic, gluconic, glutamic, isethonic, lactic, maleic, malic, mandelic, methanesulfonic, mucic, nitric, pamoic, pantothenic, phosphoric, and succinic.

While it may be possible for the compounds of the invention to be administered as the raw chemical, it is also possible to present them as a pharmaceutical formulation. Accordingly, the present invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the present invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol, Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents, Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

One example of a formulation appropriate for administration through an oral route comprises 0.60 g of the compound described in Example 6 below, 10.00 g of EtOH, 30.00 g of propylene glycol, 64.40 g of LABRAFIL® M1944 CS (oleoyl macrogol-6 glycerides EP; oleoyl polyoxyl-6 glycerides NF), and 25.00 g of LABRASOL® (caprylocaproyl macrogol-8 glycerides EP; caprylocaproyl polyoxyl-8 glycerides NF).

The compounds of the invention may be formulated for parenteral administration by injection, e.g., by bolus injection or continuous infusion. Formulations for injection may be presented in unit dosage form, e.g.; in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

One example of a formulation appropriate for administration through a parenteral route comprises 1.00 g of the compound described in Example 36 below, 30.00 g of propylene glycol, 40.00 g of CREMOPHOR® ELP (purified polyethoxylated castor oil), 10.00 g of EtOH 95%, and 19.00 g of saline solution.

In addition to the formulations described previously, the compounds of the invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compounds of the invention may take the form of tablets, lozenges, pastilles, or gels formulated in conventional manner. Such compositions may comprise the active ingredient in a flavored basis such as sucrose and acacia or tragacanth.

The compounds of the invention may also be formulated in rectal compositions such as suppositories or retention enemas, e.g., containing conventional suppository bases such as cocoa butter, polyethylene glycol, or other glycerides.

Compounds of the invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include solid, liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Via the topical route, the pharmaceutical composition according to the invention may be in the form of liquid or semi liquid such as ointments, or in the form of solid such as powders. It may also be in the form of suspensions such as polymeric microspheres, or polymer patches and hydrogels allowing a controlled release. This topical composition may be in anhydrous form, in aqueous form or in the form of an emulsion. The compounds are used topically at a concentration generally of between 0.001% and 10% by weight and preferably between 0.01% and 1% by weight, relative to the total weight of the composition.

One example of a formulation appropriate for administration through a topical route comprises 3.00 g of the compound described in Example 1 below, 35.00 g of propyleneglycol, 25.00 g of LABRASOL® (caprylocaproyl macrogol-8 glycerides EP; caprylocaproyl polyoxyl-8 glycerides NF), 15.00 g of oleic acid, 12.00 g of COMPRITOL® 888 ATO (glyceryl dibehenate EP; glyceryl behenate NF), and 10.00 g of EtOH.

The compounds of the invention presented herein may also find an application in cosmetics, in particular in body and hair hygiene and more particularly for regulating and/or restoring skin lipid metabolism.

Cosmetic use of a composition comprising, in a physiologically acceptable support, at least one of the compounds described herein for body or hair hygiene are presented. The cosmetic composition, in a cosmetically acceptable support, at least one compound and/or an optical or geometrical isomer thereof or a salt thereof, and may be in the form of liquid or semi liquid such as ointments, creams or in the form of solid such as powders. It may also be in the form of suspensions such as polymeric microspheres or polymer patches and hydrogels allowing a controlled release. This topical composition may be in anhydrous form, in aqueous form or in the form of an emulsion. The concentration of compound in the cosmetic composition is between 0.001% and 5% by weight relative to the total weight of the composition. Finally, a the present invention provides a cosmetic process for enhancing the skin, which consists in applying to the skin a composition comprising at least one compound presented herein.

For administration by inhalation, the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 m to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Compounds according to the invention can be administered at a daily dose of about 0.001 mg/kg to 100 mg/kg of body weight, in 1 to 3 dosage intakes. Further, compounds can be used systemically, at a concentration generally of between 0.001% and 10% by weight and preferably between 0.01 and 1% by weight, relative to the weight of the composition.

The amount of active ingredient that may be combined with the carrier materials to produce a single dosage form will vary depending upon the host treated and the particular mode of administration.

The compounds of the invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds of the invention described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for pain involving administration of one of the compounds described herein, increased therapeutic benefit may result by also providing the patient with another therapeutic agent for pain. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention together with inert or active compounds, or other drugs including wetting agents, flavour enhancers, preserving agents, stabilizers, humidity regulators, pH regulators, osmotic pressure modifiers, emulsifiers, UV-A and UV-B screening agents, antioxidants, depigmenting agents such as hydroquinone or kojic acid, emollients, moisturizers, for instance glycerol, PEG 400, or urea, antiseborrhoeic or antiacne agents, such as S-carboxymethylcysteine, S-benzylcysteamine, salts thereof or derivatives thereof, or benzoyl peroxide, antibiotics, for instance erythromycin and tetracyclines, chemotherapeutic agent, for example, paclitaxel, antifungal agents such as ketoconazole, agents for promoting regrowth of the hair, for example, minoxidil (2,4-diamino-6-piperidinopyrimidine 3-oxide), non-steroidal anti-inflammatory agents, carotenoids, and especially p-carotene, antipsoriatic agents such as anthralin and its derivatives, eicosa-5,8,11,14-tetraynoic acid and eicosa-5,8,11-triynoic acid, and esters and amides thereof, retinoids, i.e. RAR or RXR receptor ligands, which may be natural or synthetic, corticosteroids or oestrogens, alpha-hydroxy acids and a-keto acids or derivatives thereof, such as lactic acid, malic acid, citric acid, and also the salts, amides or esters thereof or p-hydroxy acids or derivatives thereof, such as salicylic acid and the salts, amides or esters thereof, ion-channel blockers such as potassium-channel blockers, or alternatively, more particularly for the pharmaceutical compositions, in combination with medicaments known to interfere with the immune system, anticonvulsant agents include, and are not limited to, topiramate, analogs of topiramate, carbamazepine, valproic acid, lamotrigine, gabapentin, phenytoin and the like and mixtures or pharmaceutically acceptable salts thereof. A person skilled in the art will take care to select the other compound(s) to be added to these compositions such that the advantageous properties intrinsically associated with the compounds of the invention are not, or are not substantially, adversely affected by the envisaged addition.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating diseases, disorders, conditions, or symptoms in a patient (e.g., a human or animal patient) in need of such treatment are presented herein, the methods comprising the step of administering to the patient an amount of a compound of the invention effective to reduce or prevent the disease, disorder, condition, or symptom, in combination with at least one additional agent for the treatment of said disorder that is known in the art.

In a related aspect, therapeutic compositions having at least one novel compound of the invention described herein can be administered in combination with one or more additional agents for the treatment of any of the diseases, disorders, conditions, or symptoms described herein.

### Synthetic Methods for Preparing Compounds of the Invention

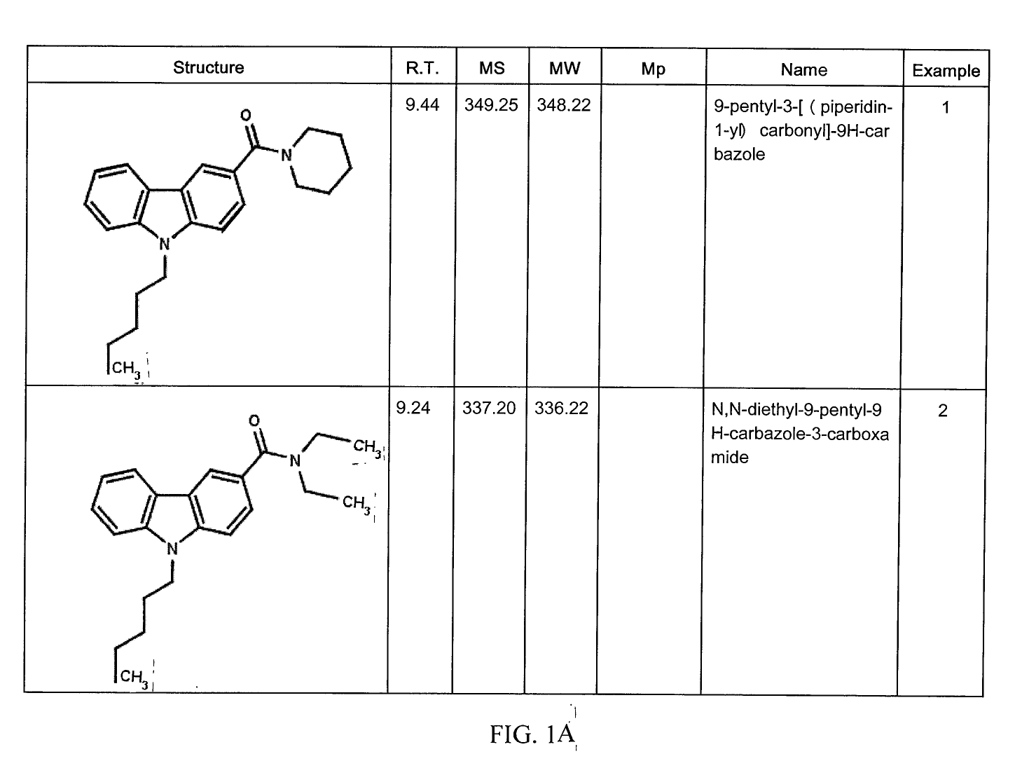

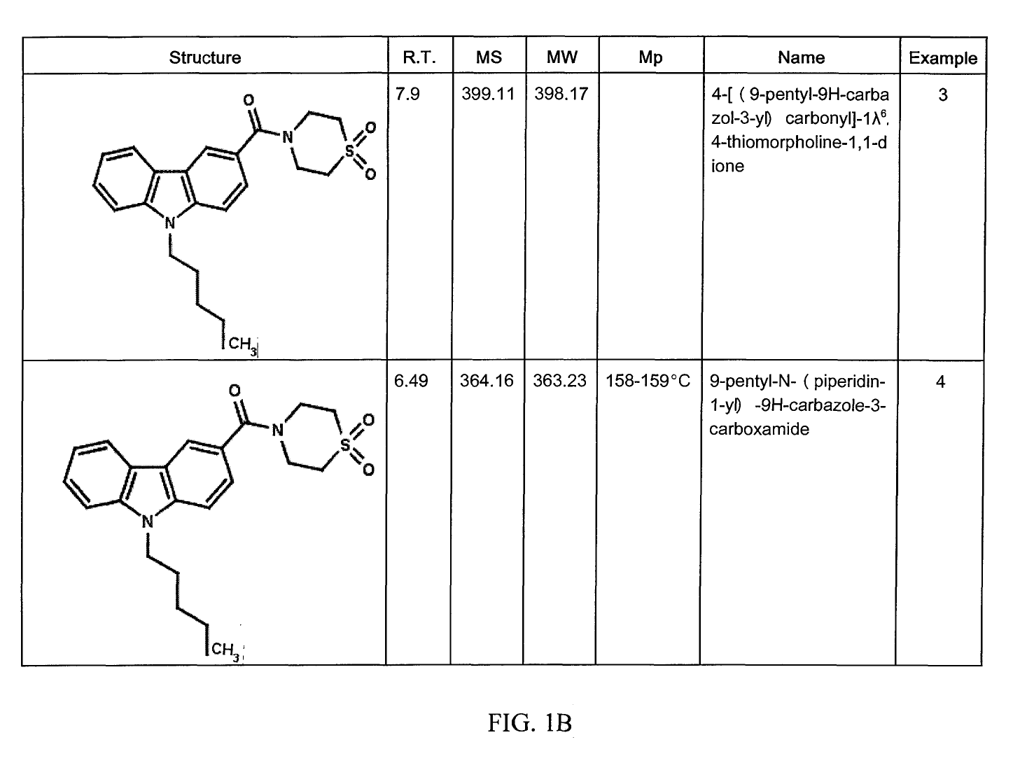

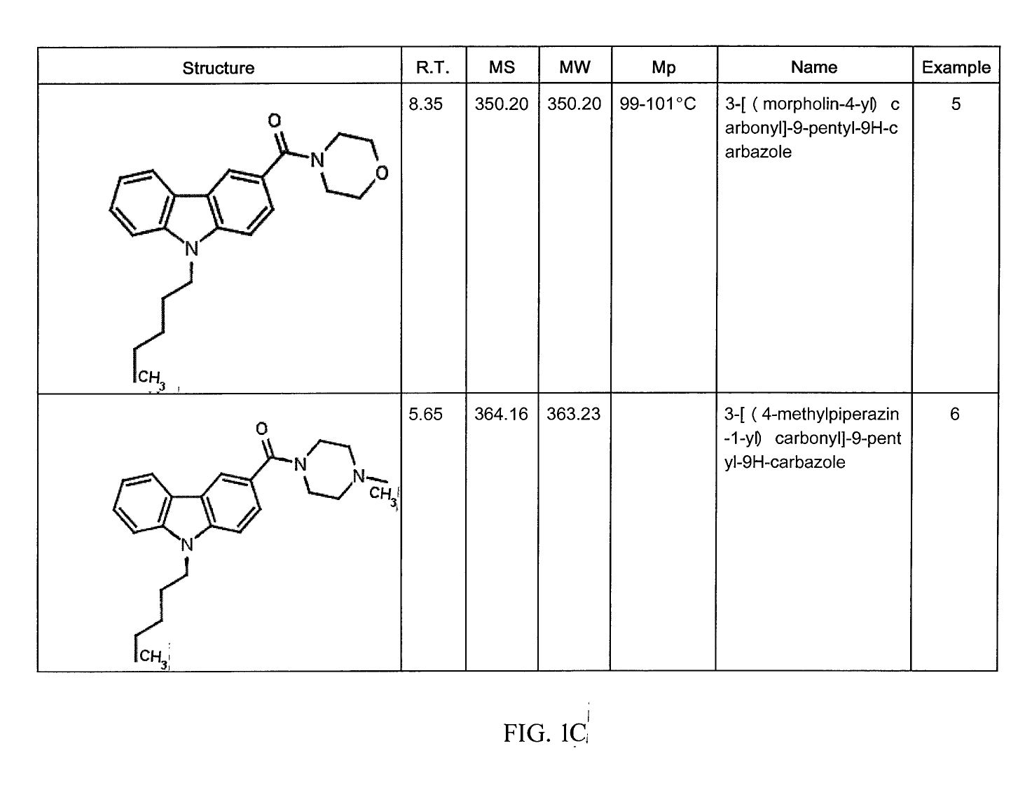

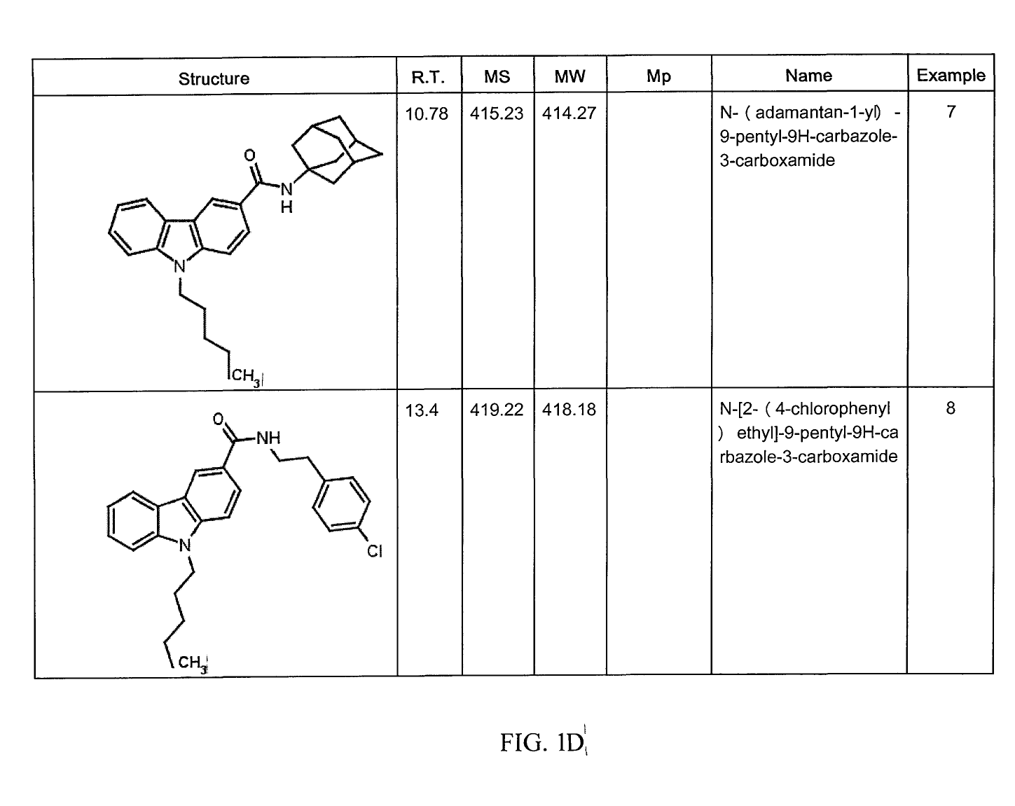

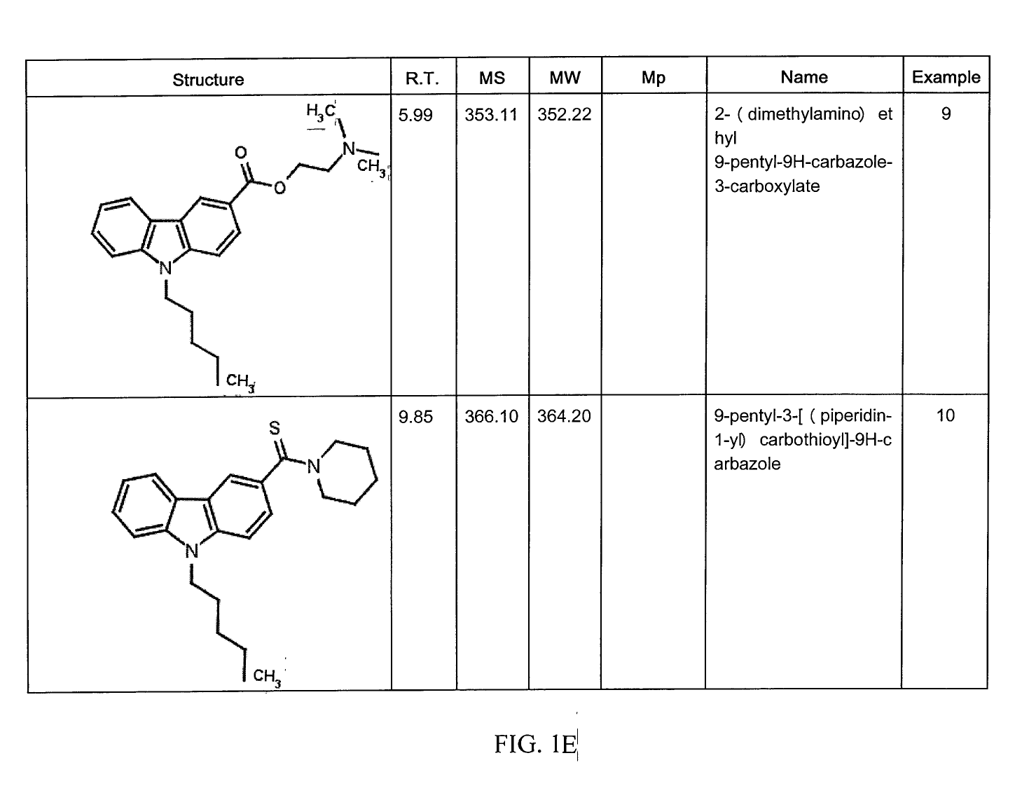

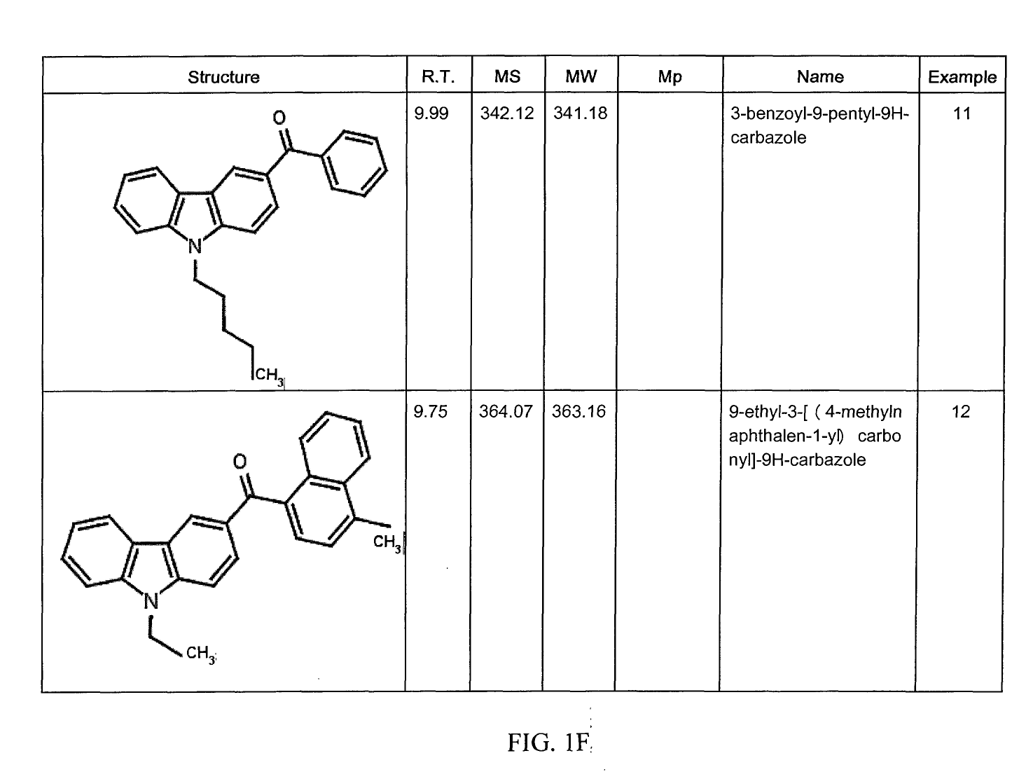

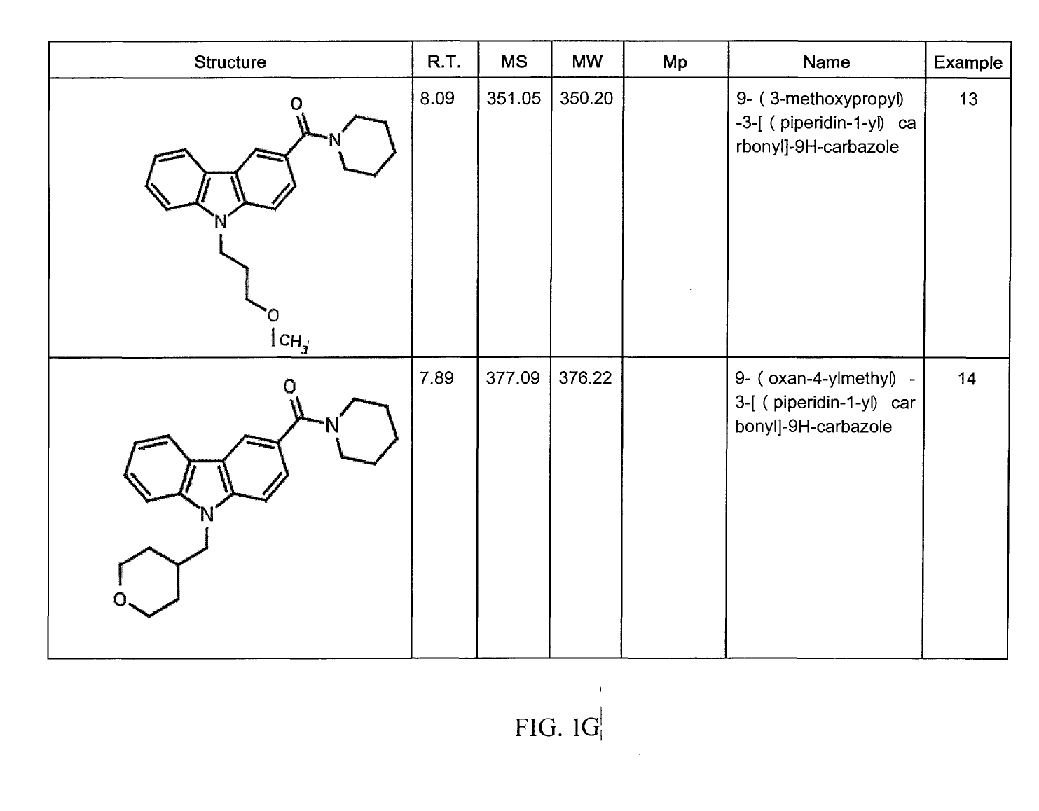

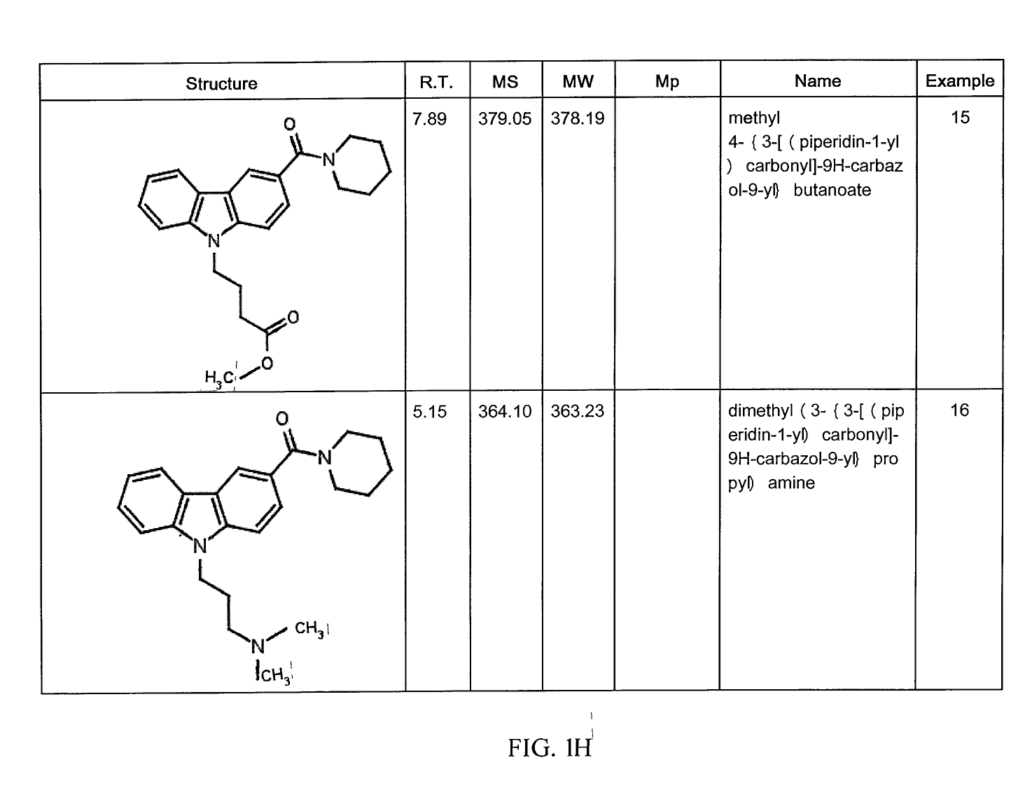

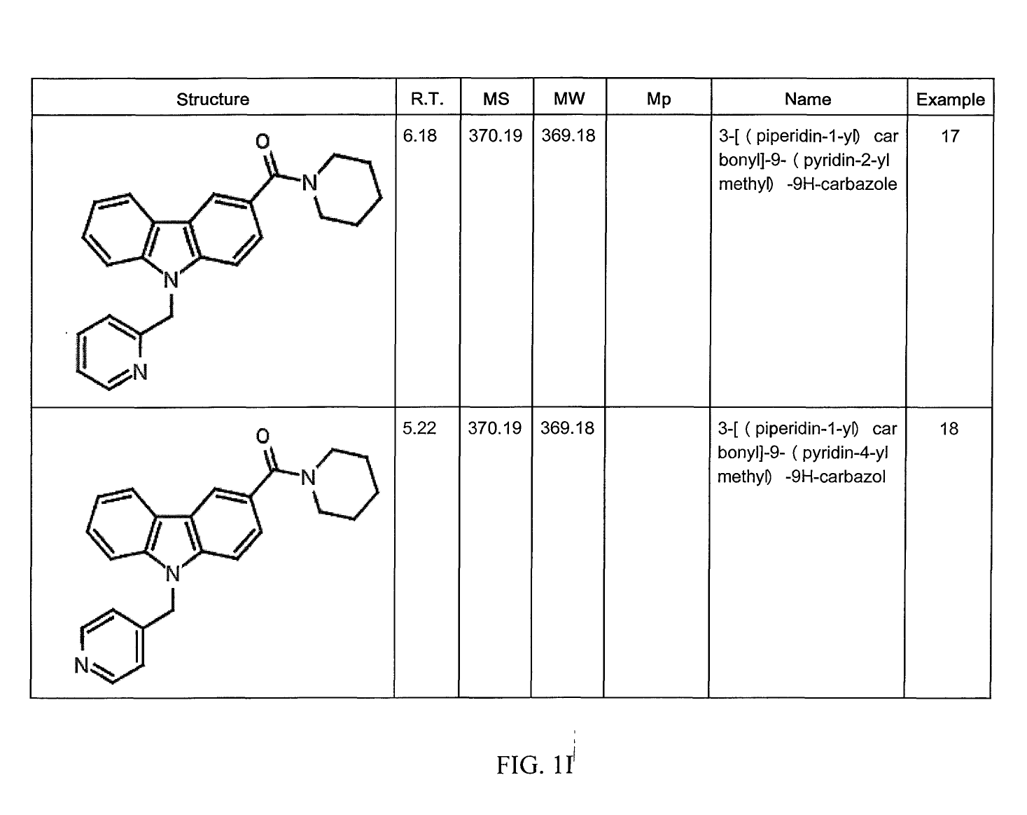

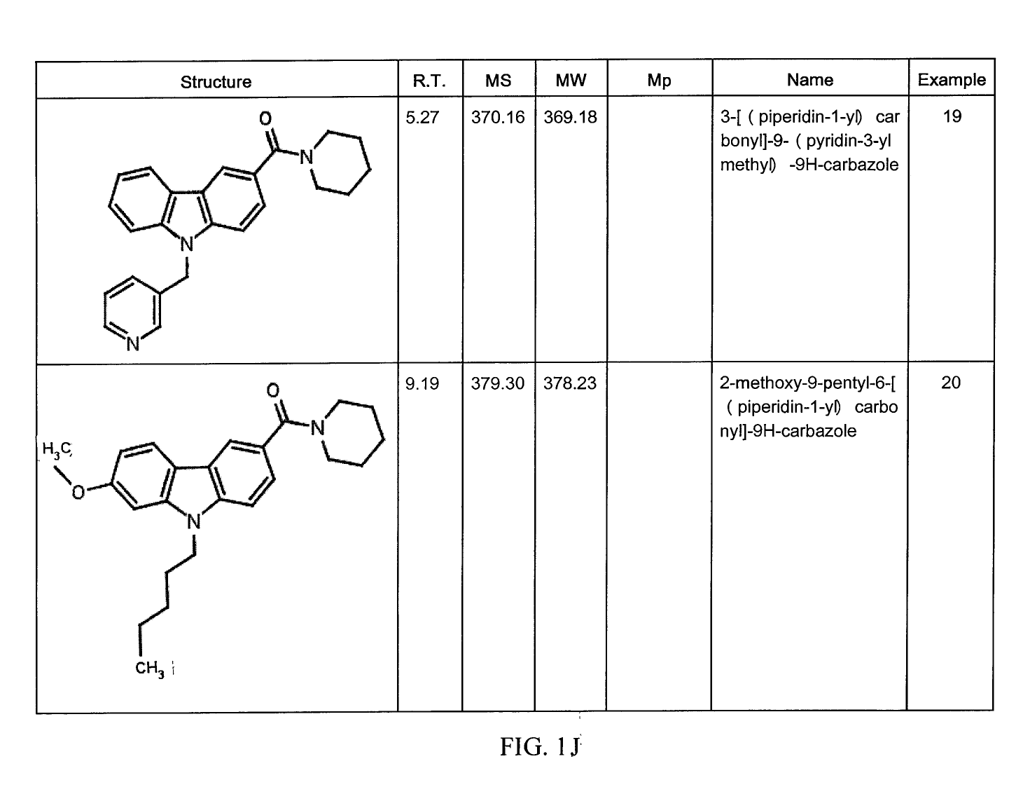

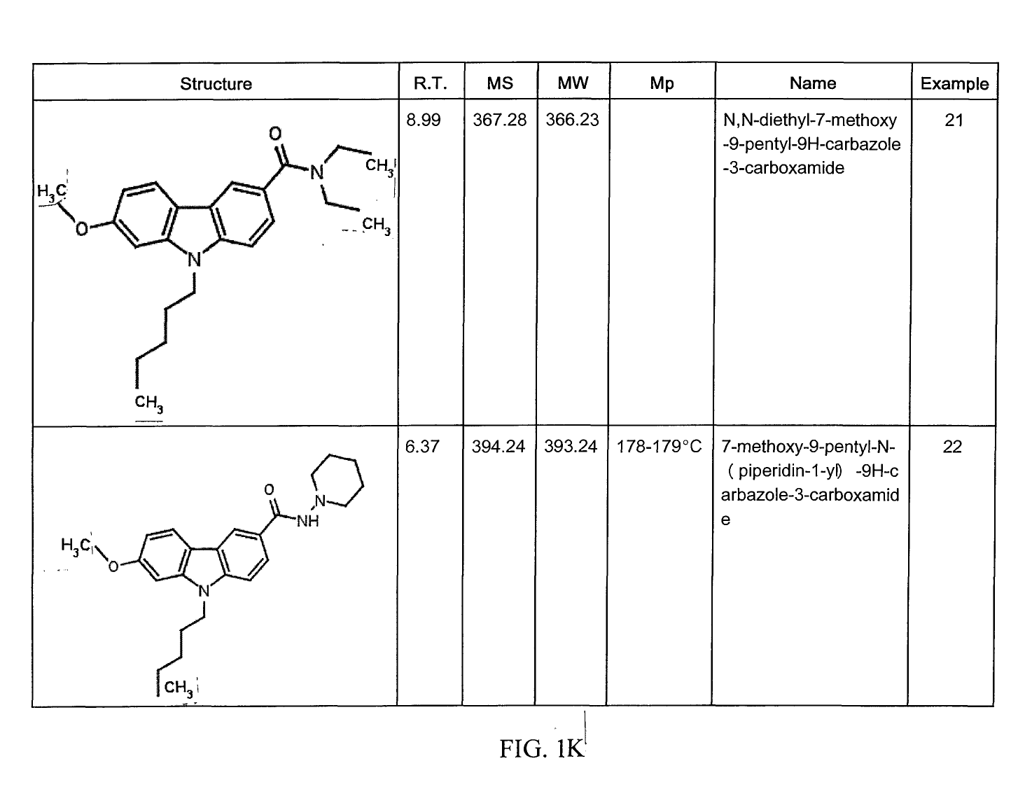

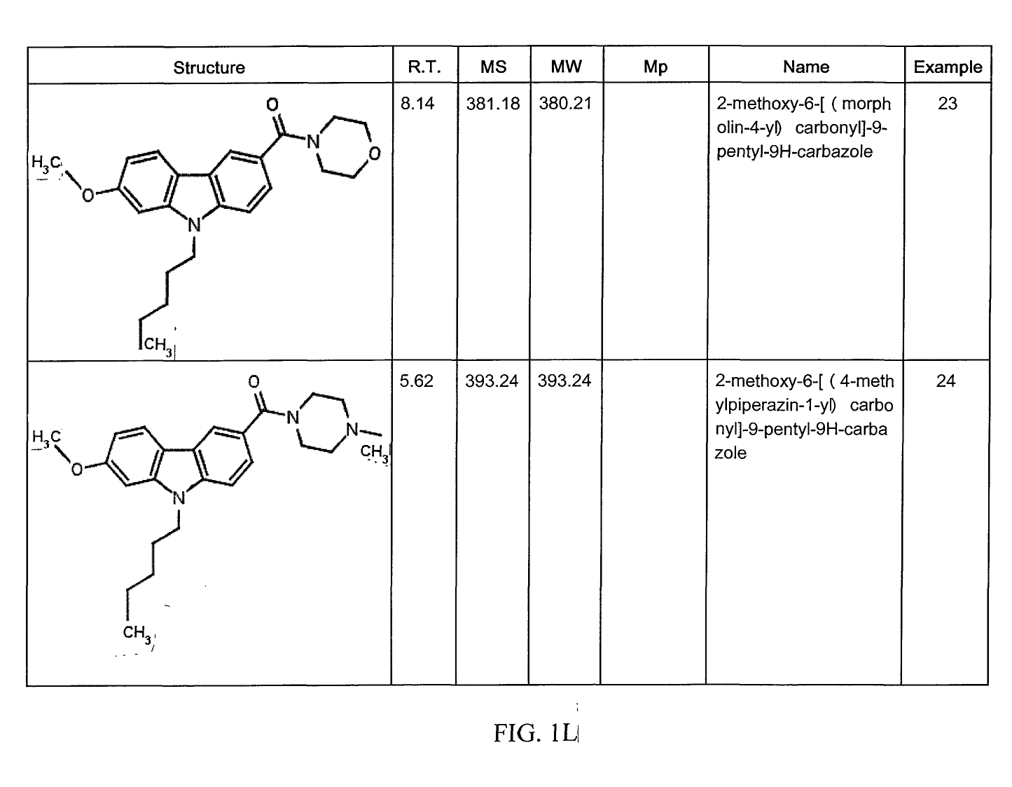

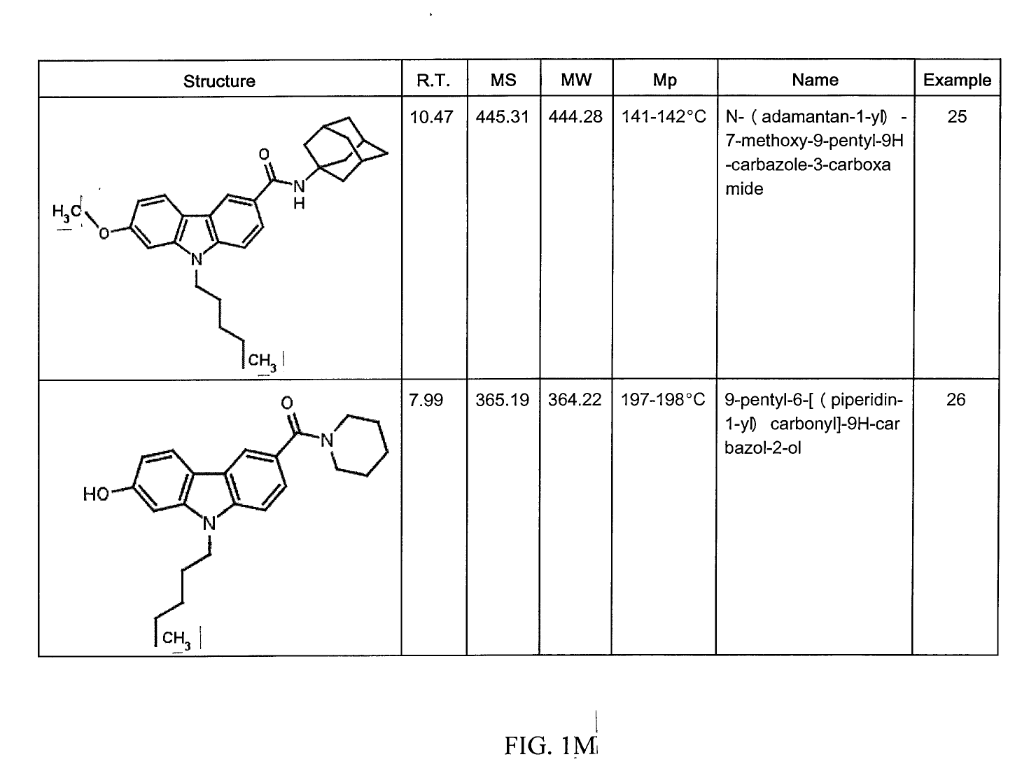

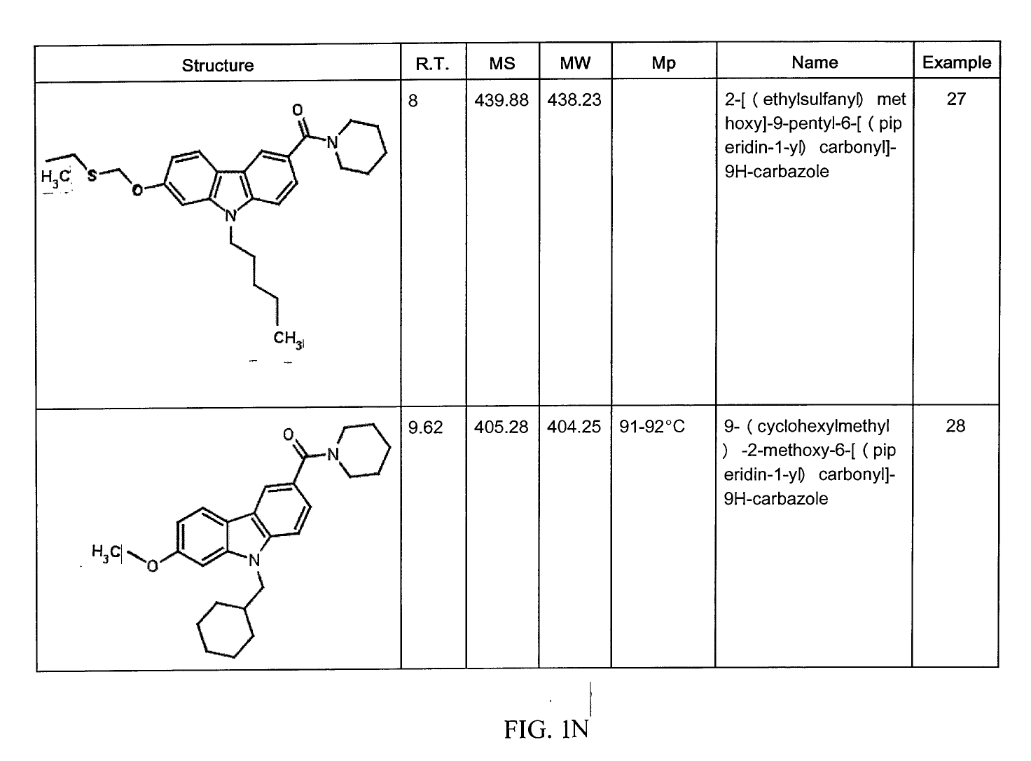

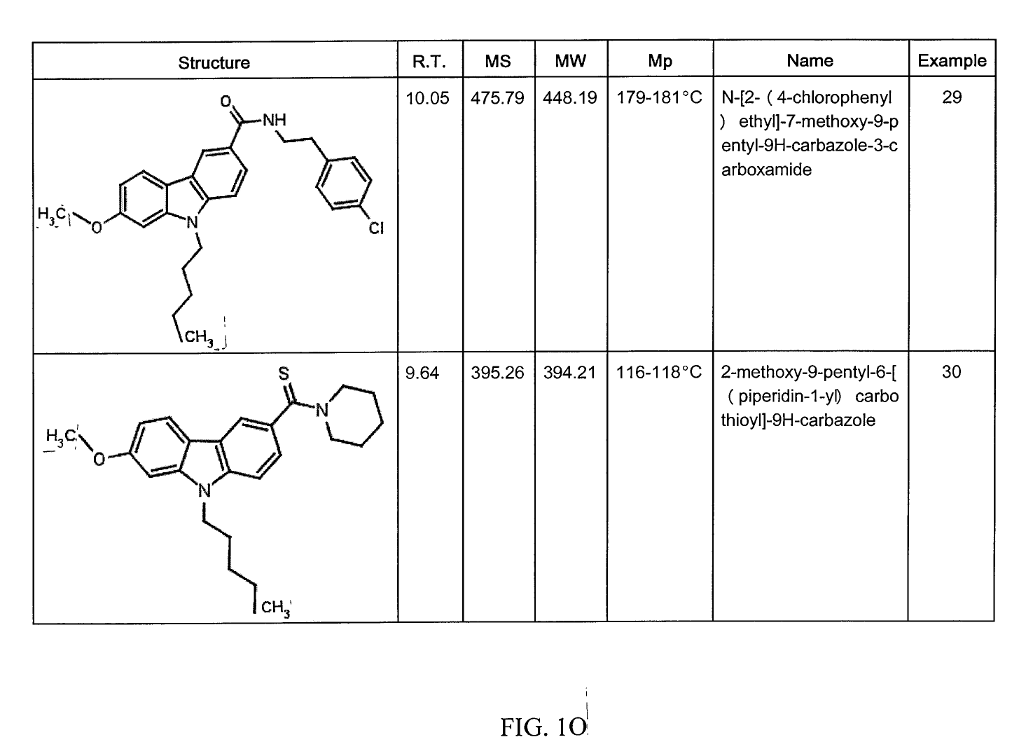

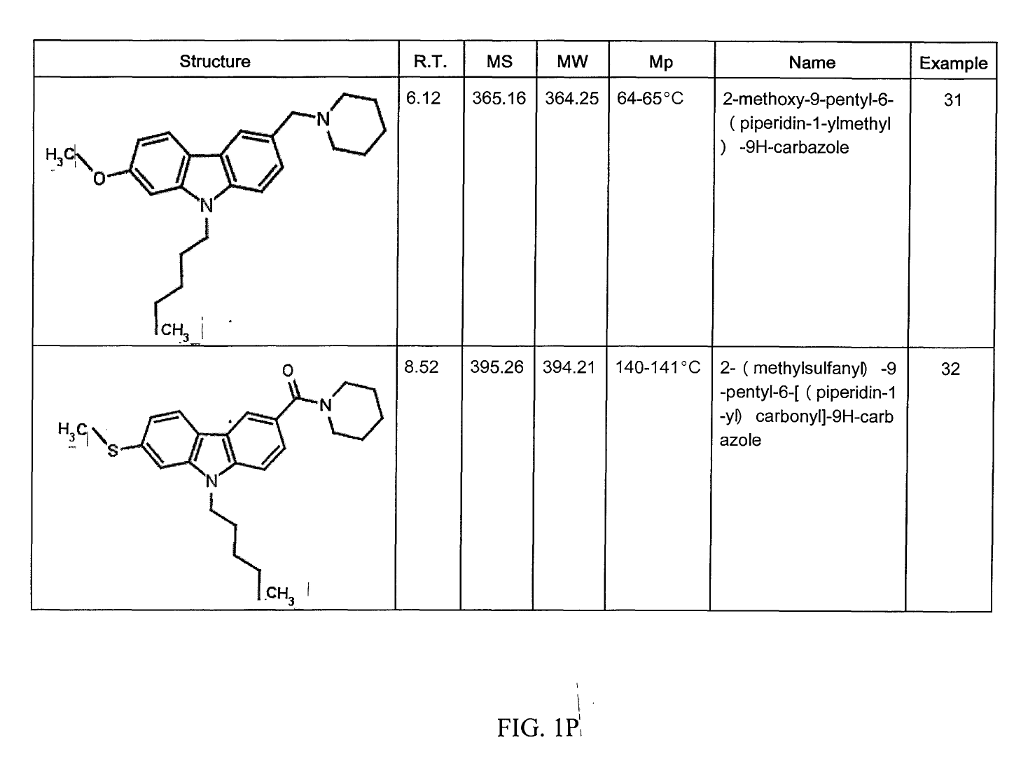

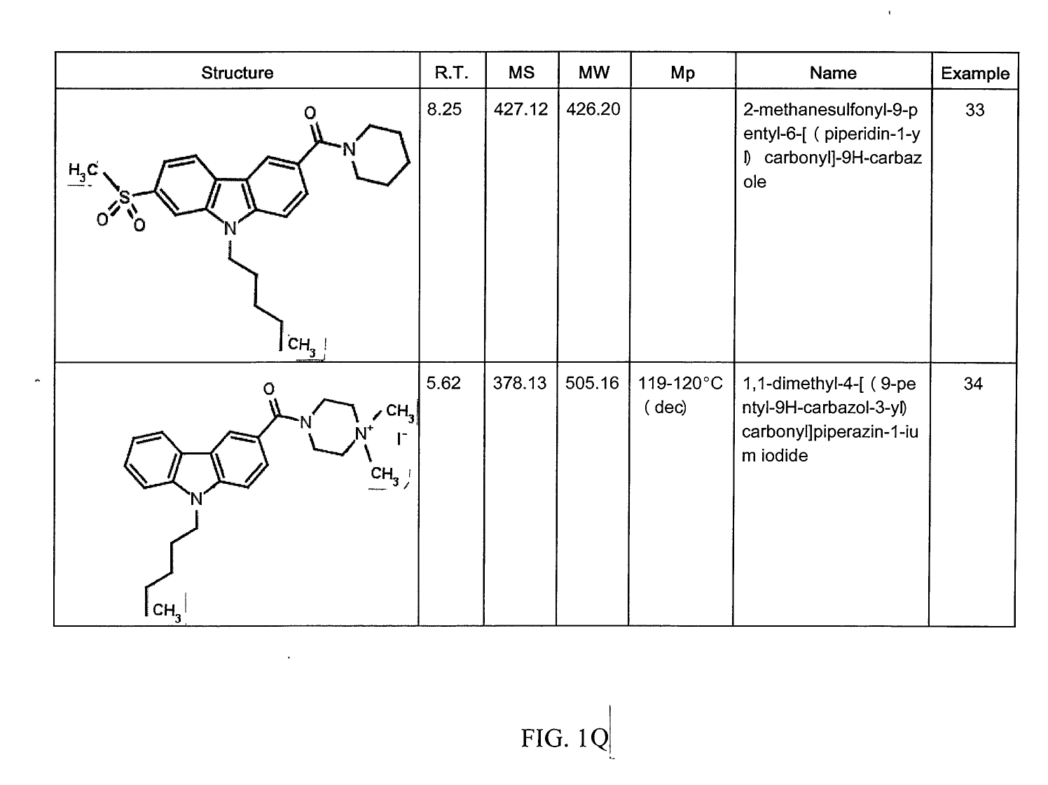

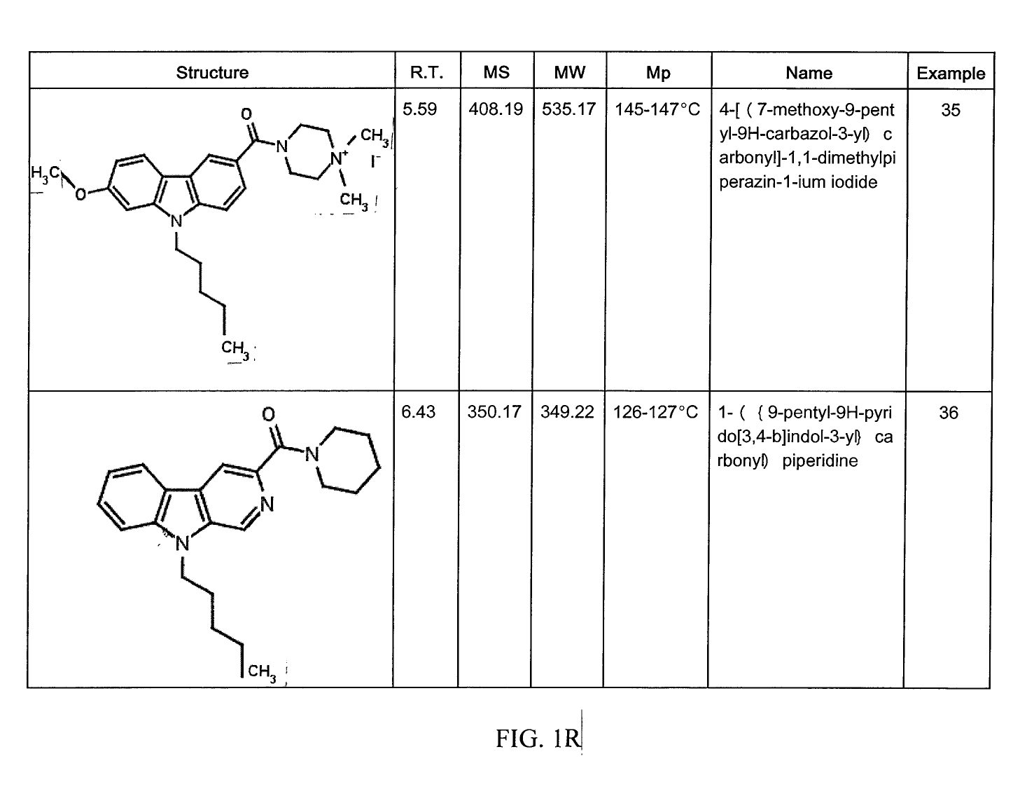

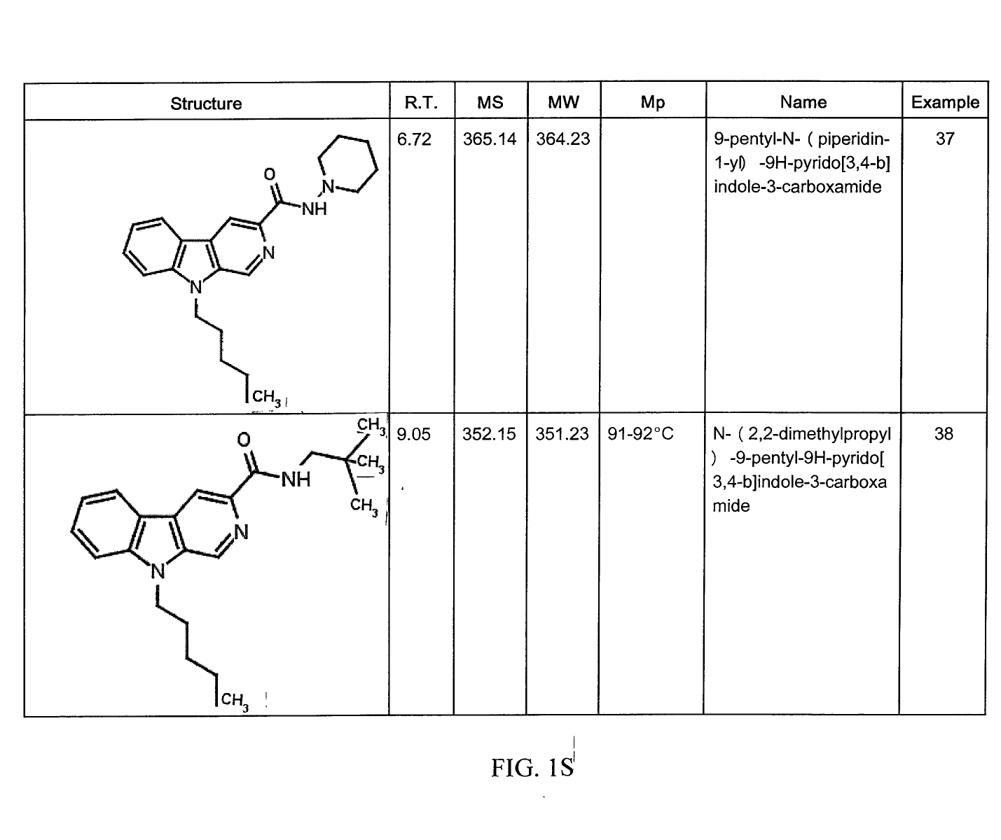

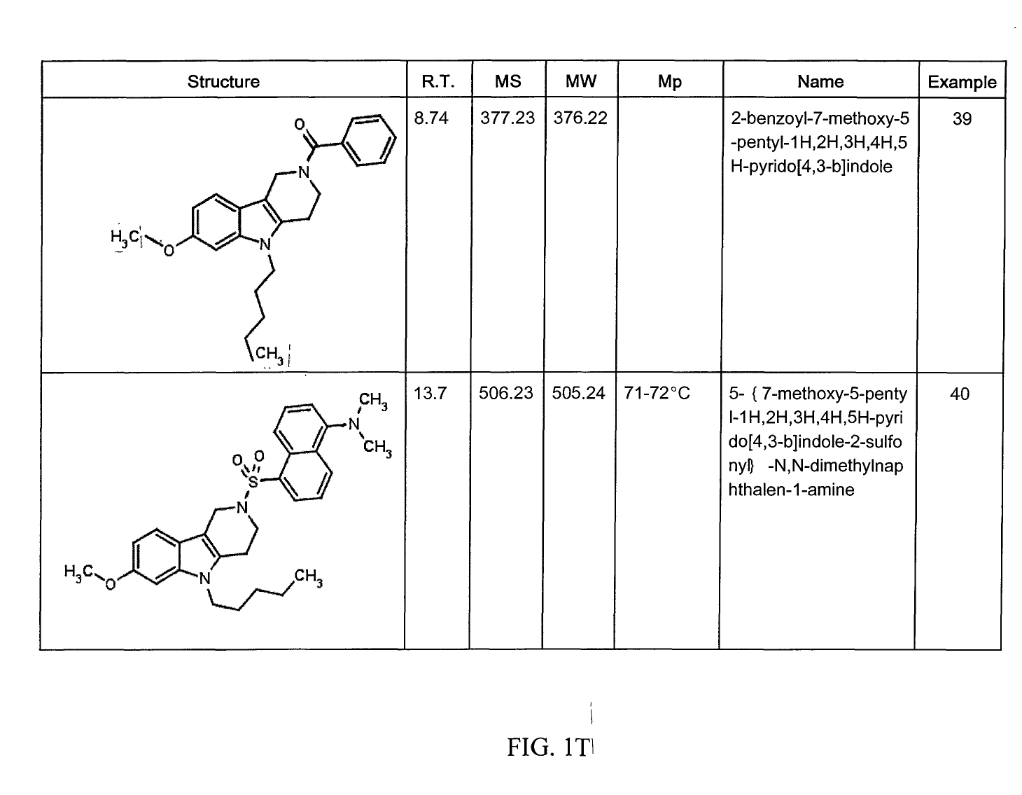

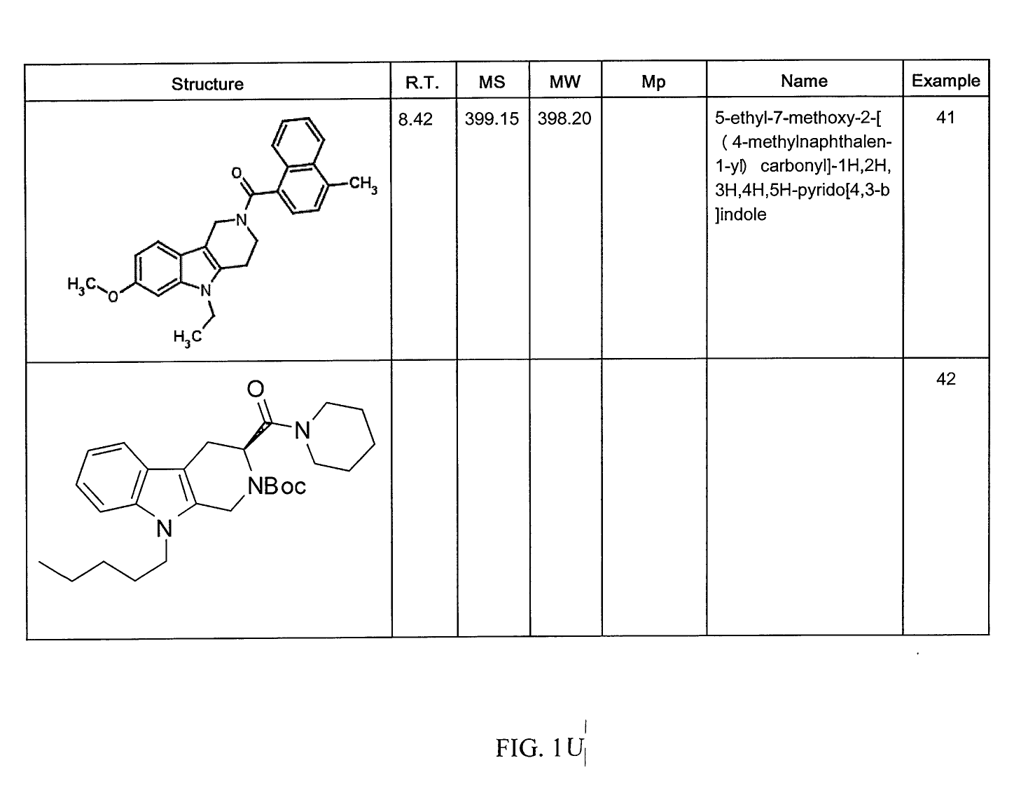

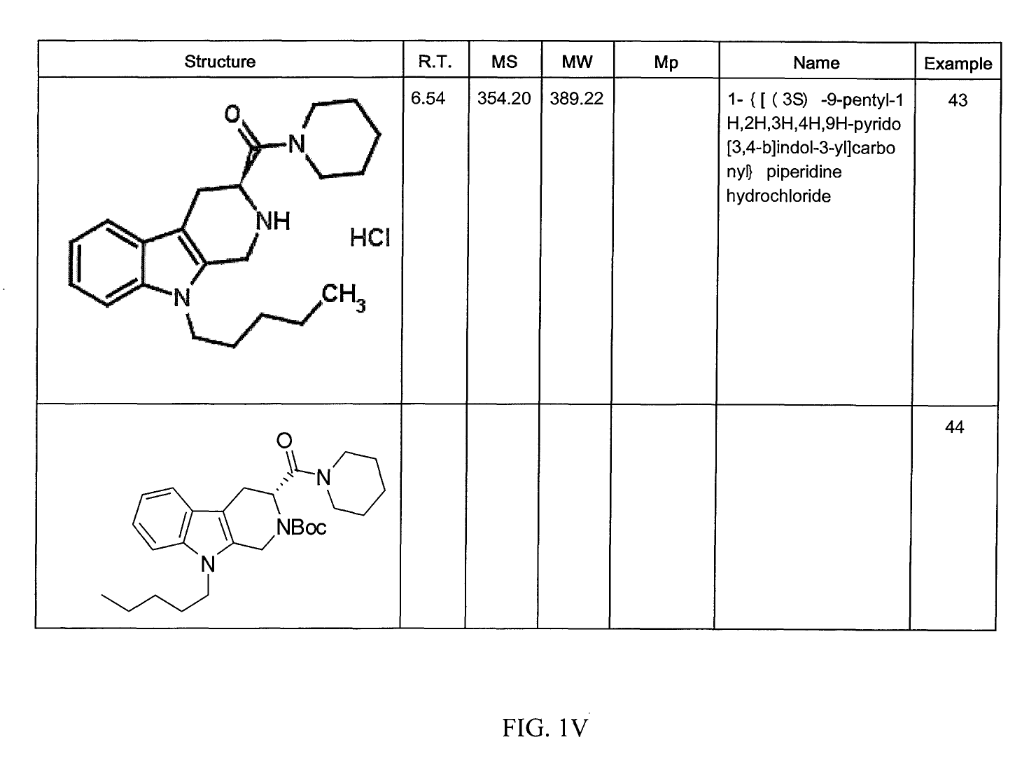

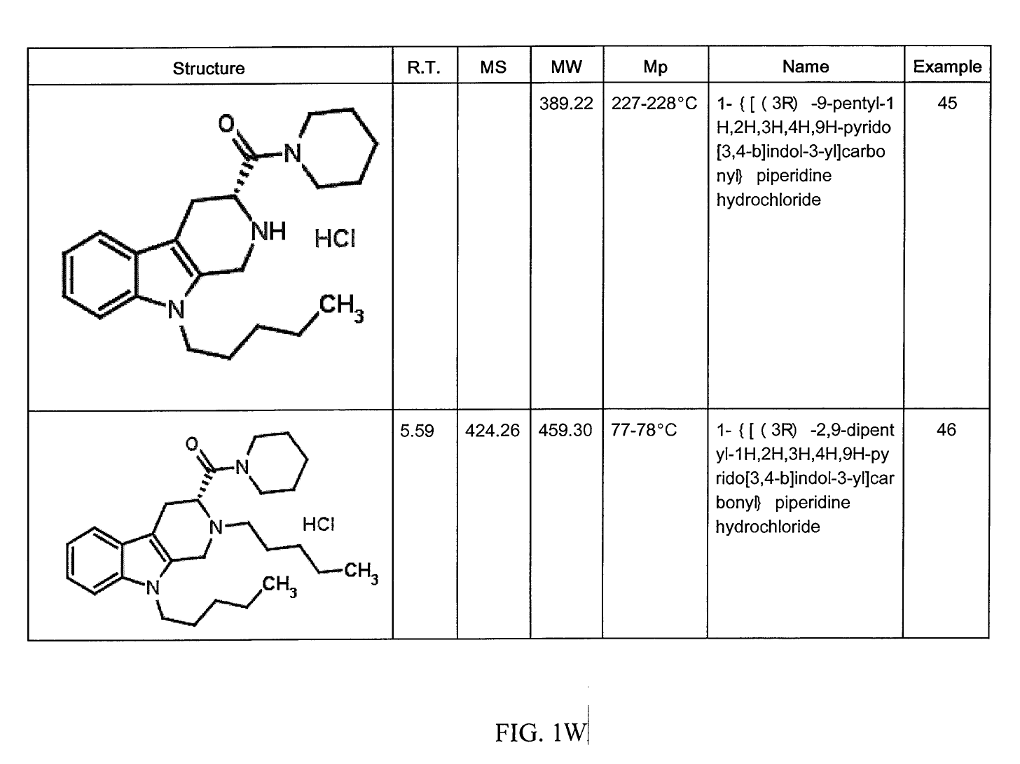

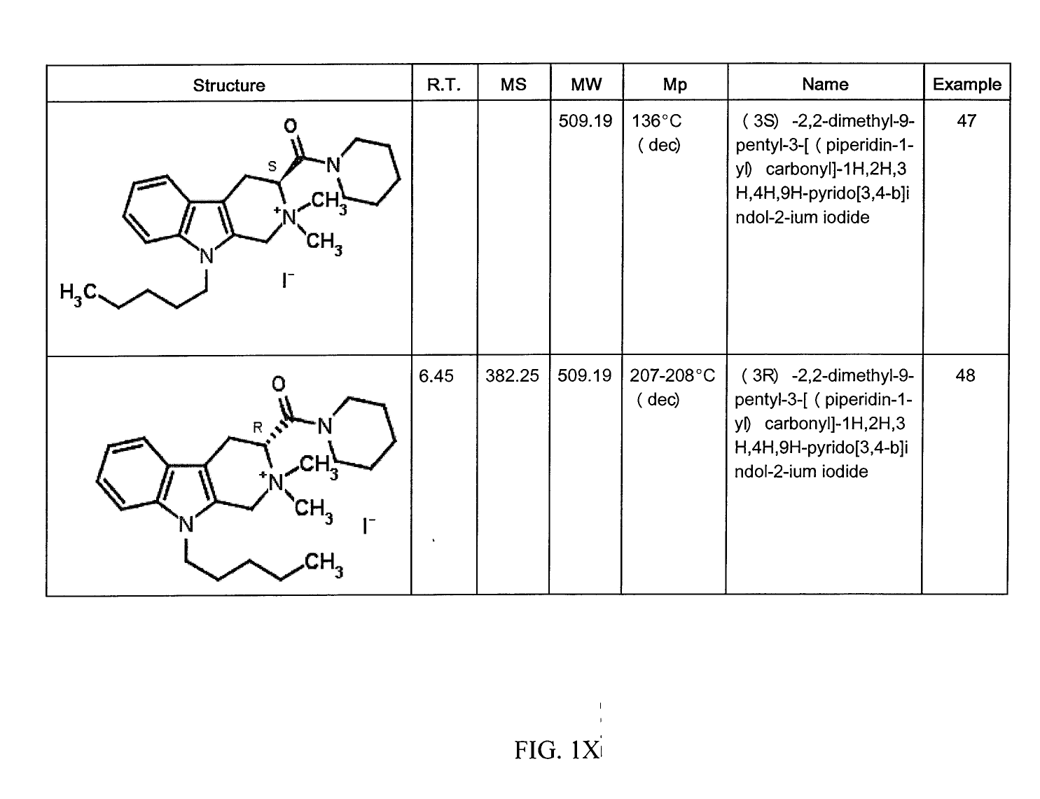

The chemical structure, name, melting point, molecular weight (theoretical and as determined by mass spectrometer), retention time, and formula of the invention classification are provided in FIG. 1 for specific embodiments of the compounds of the invention. The chemical synthesis of these species is described in detail below.

Unless otherwise stated, all reactions were carried out under a nitrogen or argon atmosphere, using commercially available anhydrous solvents. Flash column chromatography was carried out using BIOTAGE® (KP, HP, and NH) cartridges or 40-63 μm silica gel. NMR spectra were carried out on a Varian Inova 500 in the solvents specified. In the following examples, all temperatures are set uncorrected in degrees Celsius.

Abbreviations used herein: EDC (1 ethyl-3-[3-dimethylaminopropyl]carbodiimide hydrochloride), DIPEA/DIEA (N,N-diisopropyl ethyl amine), DCM (dichloromethane), DMF (dimethyl form amide), HOBT (1-hydroxybenzotriazole), BINAP (2,2′-bis(diphenylphosphino)-1,1′-binaphthyl), TFA (trifluoroacetic acid), THF (tetrahydrofuran), MeOH (methanol), Pd(OAc)₂ (palladium acetate) K₂CO₃ (potassium carbonate), Cs₂CO₃ (cesium carbonate), Mg₂SO₄ (magnesium sulfate), NaHCO₃ (sodium bicarbonate), KOt-Bu (potassium tert-butoxide), HCl (hydrochloric acid), NaOH (sodium hydroxide), KMnO₄ (potassium permanganate), brine (saturated aqueous sodium chloride solution), AlCl₃ (aluminum trichloride), LAH (lithium aluminium hydride), EtOAc (ethyl acetate), CHCl₃ (chloroform), DMAP (4-(dimethylamino)pyridine), celite (diatomaceous earth), EtOH (ethanol), TBAI (tetrabutyl ammonium iodide), TLC (thin layer chromatography), NMR (nuclear magnetic resonance), DMSO-d₆ (deuterated dimethyl sulfoxide), CDCl₃ (deuterated chloroform), LC-MS (LC-MS liquid chromatography-mass spectrometry), HPLC (high pressure liquid chromatography or high performance liquid chromatography), SAR (structure-activity relationships), DI (deionized).

### Example 1: (9-pentyl-9H-carbazol-3-yl)(piperidin-1-yl)methanone (compound 4)

### Synthesis of 9-pentyl-9H-carbazole (compound 1)

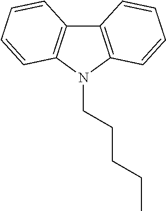 [SMILES: CCCCCn1c2ccccc2c2ccccc21]

Under argon atmosphere, a solution of carbazole (2.5 g, 14.95 mmol), 1-bromopentane (2.225 mL, 17.94 mmol), and Cs₂CO₃ (7.3 g, 22.41 mmol) in DMF (20 mL) was subjected to microwave irradiation at 140° C. for 1 h. The reaction mixture was cooled, diluted with ethyl acetate (50 mL), and filtered. The organic solvents were evaporated in vacuo. The resultant dark oil was distilled under reduced pressure (125° C., 2 mmHg) to afford the title compound as yellowish oil (3.169 g, 89%).

### Synthesis of 9-pentyl-9H-carbazole-3-carbaldehyde (compound 2)

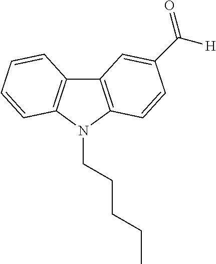 [SMILES: CCCCCn1c2ccccc2c2cc(C=O)ccc21]

The title compound was prepared according to a modified literature procedure (Pajda et al., Modern Polymeric Materials for Environmental Applications 129 (2006)) POCl₃ (2.6 mL, 28.40 mmol) was added, over a period of 10 min., to an ice-cooled, stirred DMF (7.43 mL, 96 mmol) under nitrogen. The reddish solution was allowed to stir at room temperature for 1 h. Carbazole 1 (3.169 g, 13.35 mmol) was added over 10 min., and the obtained mixture was subjected to microwave irradiation at 100° C. for 1 h. The reaction mixture was cooled and then poured into crushed ice. After warming to room temperature, the resultant product was extracted into ethyl acetate, and the organic phase was washed with water, brine, dried (MgSO₄), filtered, and evaporated in vacuo. The obtained residue was purified by column chromatography on silica gel using heptanes/ethyl acetate in different proportions to afford the title compound as a white solid (3.49 g, 99%).

### Synthesis of 9-pentyl-9H-carbazole-3-carboxylic acid (compound 3)

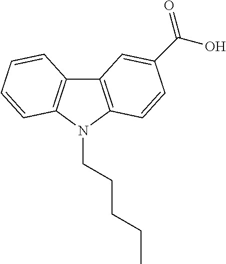 [SMILES: CCCCCn1c2ccccc2c2cc(C(=O)O)ccc21]

To a cold solution (ice-water bath) of 9-pentyl-3-formylcarbazole (2.96 g, 11.16 mmol) in water/acetone (100 mL, 1:1. v/v) was added dropwise with stirring a solution of potassium permanganate (1.8 g, 11.39 mmol) in acetone (50 mL). The mixture was heated 5 h at reflux and allowed to cool to room temperature. The mixture was filtered through a pad of celite and concentrated in vacuo to remove acetone. The obtained solution was diluted with water (100 mL), basified with NaOH to pH ca. 10, and extracted with heptane/ether (4:1, v/v, 50 mL×3) to remove the unreacted starting material. The aqueous solution was cooled on an ice-water bath and acidified with ice-cold solution of sulfuric acid (20%) to pH ca. 2. The resultant bulky precipitate was extracted into ethyl acetate and the extract was washed with brine, dried over magnesium sulfate, filtered, and concentrated in vacuo. The precipitated product was collected by filtration, washed several times with heptanes, and dried overnight to produce the title compound 3 (2.743 g, 87%) as a greenish solid.

### (9-pentyl-9H-carbazol-3-yl)(piperidin-1-yl)methanone (compound 4)

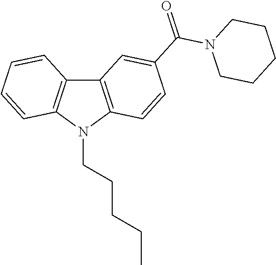 [SMILES: CCCCCn1c2ccccc2c2cc(C(=O)N3CCCCC3)ccc21]

9H-carbazole-3-carboxylic acid 3 (300 mg, 1.07 mmol), piperidine (215 mg, 2.53 mmol), DIPEA (363 μL, 2.14 mmol), and DMAP) (156 mg, 1.28 mmol) were added to DCM (30 mL) under nitrogen. The obtained solution was cooled down on an ice-water bath, EDC (350 mg, 1.83 mmol) was added to the solution, and the reaction mixture was then allowed to warm to room temperature and stirred for 16 h. The solvent was removed in vacuo, and the obtained residue was extracted into ethyl acetate (100 mL). The organic layer was washed consecutively with 5% citric acid solution (50 mL×3), concentrated sodium bicarbonate (50 mL×3), brine (50 mL), dried over magnesium sulfate, filtered, and concentrated in vacuo. The residue was purified on silica gel using heptanes/ethyl acetate in different proportions to afford the title compound as a yellowish glass (345 mg, 93%).

### Example 2: Synthesis of N,N-diethyl-9-pentyl-9H-carbazole-3-carboxamide (compound 5)

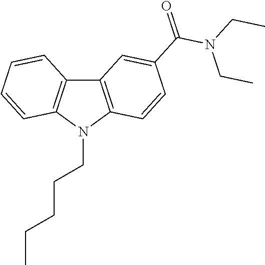 [SMILES: CCCCCn1c2ccccc2c2cc(C(=O)N(CC)CC)ccc21]

Using 9H-carbazole-3-carboxylic acid 3 (112 mg, 0.40 mmol) and diethylamine (74 μL, 0.71 mmol) as starting compounds, the title compound was prepared following the procedures described in preparation of compound 4. A colorless viscous oil was obtained. Yield: 44 mg (33%).

### Example 3: Synthesis of (9-pentyl-9H-carbazol-3-yl)(1,1-dioxo-thiomorpholino)methanone (compound 6)

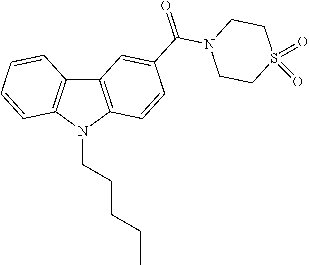 [SMILES: CCCCCn1c2ccccc2c2cc(C(=O)N3CCS(=O)(=O)CC3)ccc21]

Using 9H-carbazole-3-carboxylic acid 3 (115 mg, 0.41 mmol) and 1,1-dioxo-thiomorpholine (80 mg, 0.59 mmol) as starting compounds, the title compound was prepared following the procedures described in preparation of compound 4. A yellowish glass was obtained. Yield: 83 mg, (51%).

### Example 4: Synthesis of 9-pentyl-N-(piperidin-1-yl)-9H-carbazole-3-carboxamide (compound 7)

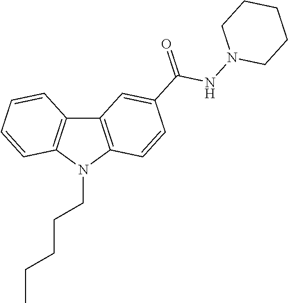 [SMILES: CCCCCn1c2ccccc2c2cc(C(=O)NN3CCCCC3)ccc21]

Using 9H-carbazole-3-carboxylic acid 3 (100 mg, 0.36 mmol) and 1-aminopiperidine (39 μL, 0.36 mmol) as starting compounds, the title compound was prepared following the procedures described in preparation of compound 4. A yellowish solid. Yield: 109 mg (84%); nip 158-159° C.

### Example 5: Synthesis of morpholino(9-pentyl-9H-carbazol-3-yl)methanone (compound 8)

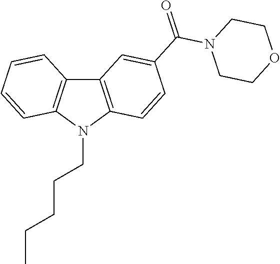 [SMILES: CCCCCn1c2ccccc2c2cc(C(=O)N3CCOCC3)ccc21]

Using 9H-carbazole-3-carboxylic acid 3 (100 mg, 0.36 mmol) and morpholine (62 μL, 0.71 mmol) as starting compounds, the title compound was prepared following the procedures described in preparation of compound 4. A white solid was obtained. Yield: 109 mg (84%); mp 99-101° C.

### Example 6: Synthesis of (4-methylpiperazin-1-yl)(9-pentyl-9H-carbazol-3-yl)methanone (compound 9)

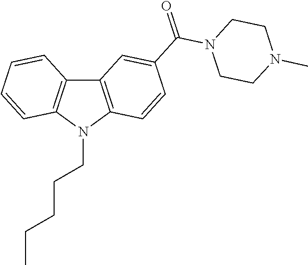 [SMILES: CCCCCn1c2ccccc2c2cc(C(=O)N3CCN(C)CC3)ccc21]

Using 9H-carbazole-3-carboxylic acid 3 (110 mg, 0.39 mmol) and 1-methylpiperazine (71 mg, 0.71 mmol) as starting compounds, the title compound was prepared following the procedures described in preparation of compound 4. An orange viscous oil was obtained. Yield: 134 mg (94%).

### Example 7: Synthesis of N-(1-adamantyl)-9-pentyl-9H-carbazole-3-carboxamide (compound 10)

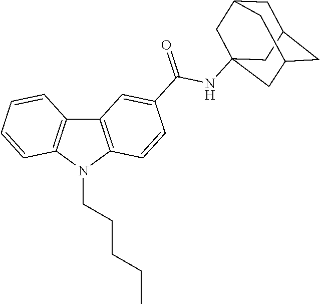 [SMILES: CCCCCn1c2ccccc2c2cc(C(=O)NC34CC5CC(CC(C5)C3)C4)ccc21]

Using 9H-carbazole-3-carboxylic acid 3 (112 mg, 0.40 mmol) and 1-adamantylamine (74 μL, 0.71 mmol) as starting compounds, the title compound was prepared following the procedures described in preparation of compound 4. A yellowish glass was obtained. Yield: 91 mg (62%).

### Example 8: Synthesis of N-(4-chlorophenethyl)-9-pentyl-9H-carbazole-3-carboxamide (compound 11)

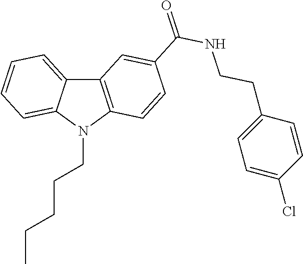 [SMILES: CCCCCn1c2ccccc2c2cc(C(=O)NCCc3ccc(Cl)cc3)ccc21]

Using 9H-carbazole-3-carboxylic acid 3 (105 mg, 0.37 mmol) and 2-(4-chlorophenyl)ethanamine (90 mg, 0.58 mmol) as starting compounds, the title compound was prepared following the procedures described in preparation of compound 4. An off-white solid was obtained. Yield: 38 mg (23%).

### Example 9: Synthesis of 2-(dimethylamino)ethyl 9-pentyl-9H-carbazole-3-carboxylate (compound 12)

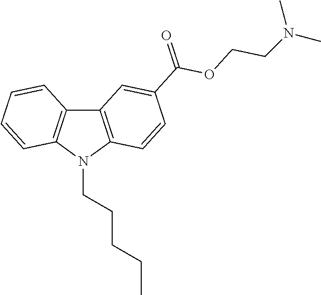 [SMILES: CCCCCn1c2ccccc2c2cc(C(=O)OCCN(C)C)ccc21]

Carboxylic acid 1 (100 mg, 0.36 mmol), 2-(dimethylamino)ethanol (36 μL, 0.36 mmol), and DMAP (87 mg, 0.71 mmol) were added to DCM (20 mL) under nitrogen. EDC (200 mg, 1.04 mmol) was added to the solution, and the reaction mixture was stirred for 16 h. The solvent was removed in vacuo, and the obtained residue was extracted into ethyl acetate (100 mL). The organic layer was washed consecutively with concentrated sodium bicarbonate (50 mL 3), brine (50 mL), dried over magnesium sulfate, filtered, and concentrated in vacuo. The residue was purified on a BIOTAGE® KP-NH cartridge (amino-modified silica gel) using heptanes/ethyl acetate in different proportions to afford the title compound as a yellowish viscous oil (117 mg, 93%).

### Example 10: 9-pentyl-9H-carbazol-3-yl)(piperidin-1-yl)methanethione (compound 13)

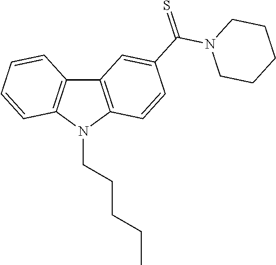 [SMILES: CCCCCn1c2ccccc2c2cc(C(=S)N3CCCCC3)ccc21]

Under argon atmosphere, a solution of carbazole 4 (60 mg, 0.17 mmol) and Lawesson's reagent (49 mg, 0.12 mmol) in toluene (3 mL) was tightly capped in a 5 mL microwave vessel. The mixture was subjected to microwave irradiation at 140° C. for 4 h and then cooled to room temperature. The organic solvent was evaporated in vacuo, and the residue was purified by column chromatography on silica gel using heptanes/ethyl acetate in different proportions to yield thioamide 13 as a yellow glass. Yield: 48 mg (76%).

### Example 11: (9-pentyl-9H-carbazol-3-yl)(phenyl)methanone (compound 14)

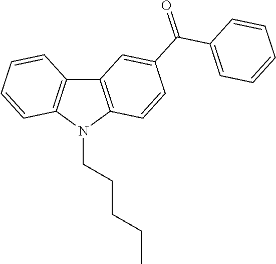 [SMILES: CCCCCn1c2ccccc2c2cc(C(=O)c3ccccc3)ccc21]

Under argon atmosphere, AlCl₃ (309 mg, 2.32 mmol) was added to a solution of carbazole 1 (500 mg, 2.11 mmol) in dry benzene (30) mL, and the obtained solution was placed in an ice-water bath for 20 min. Benzoyl chloride (282 μL, 2.43 mmol) was added dropwise via a syringe to the solution, and the reaction mixture was then allowed to warm to room temperature and stirred for 16 h. The reaction mixture was cooled on an ice-water bath then poured onto a mixture of ice and concentrated NaOH and extracted with diethyl ether. The organic phase was washed with saturated aqueous sodium bicarbonate, brine, dried (MgSO₄), filtered and evaporated in vacuo. The obtained residue was purified by column chromatography on silica gel eluting with ethyl acetate/heptanes in different proportions to give compound 14 (514 mg, 71%) as a yellowish solid: mp 116-117° C.

### Example 12: Synthesis of (9-ethyl-9H-carbazol-3-yl)(4-methylnaphthalen-1-yl)methanone (compound 16)

### Synthesis of 9-ethyl-9H-carbazole (compound 15)

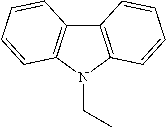

A mixture of carbazole (10 g, 59.80 mmol), ethyl bromide (6.65 mL, 89.75 mmol), and powdered NaOH (4 g, 100 mmol) in dry acetone (100 mL) was refluxed for 16 h under nitrogen. The organic solvents were evaporated in vacuo. The obtained residue was diluted with water (50 mL) and extracted into tert-butyl methyl ether (100 mL). The organic layer was washed with water, brine, dried (MgSO4), filtered, and evaporated in vacuo. The obtained residue was crystallized from ethanol. Yield: 8.62 g (74%); mp 70-71° C.

### Synthesis of (9-ethyl-9H-carbazol-3-yl)(4-methylnaphthalen-1-yl)methanone (compound 16)

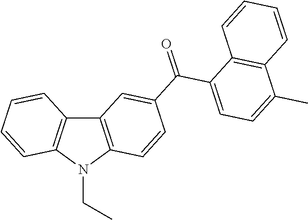 [SMILES: CCn1c2ccccc2c2cc(C(=O)c3ccc(C)c4ccccc34)ccc21]

Using carbazole 15 (426 mg, 2.18 mmol) and 4-methyl-1-naphthoyl chloride (Huffman et al., Bioorganic & Medicinal Chemistry 13:89 (2005)) (487.87 mg, 2.62 mmol) as starting compounds, the title compound was prepared following the procedures described in preparation of compound 14 as a yellow glass. Yield: 427 mg (54%).

### Example 13: Synthesis of (9-(3-methoxypropyl)-9H-carbazol-3-yl)(piperidin-1-yl)methanone (compound 22)

### Synthesis methyl 4-(phenylamino)benzoate (compound 17)

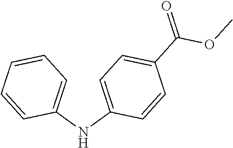 [SMILES: COC(=O)c1ccc(Nc2ccccc2)cc1]

Under argon atmosphere, a solution of methyl 4-bromobenzoate (3.5 g, 16.28 mmol), aniline (1.819 g, 1953, mmol), palladium (II) acetate (218 mg, 0.97 mmol), rac-BINAP (506 mg, 0.81 mmol), and potassium carbonate (6.72 g, 48.62 mmol) in toluene (ca. 10 mL) was tightly capped in a 25 mL microwave vessel. The mixture was subjected to microwave irradiation at 160° C. for 2 h and then cooled to room temperature. The reaction mixture was diluted with DCM and filtered. The organic solvents were evaporated in vacuo, and the residue was suspended in methyl tert-butyl ether (150 mL). The organic phase was washed with saturated aqueous sodium bicarbonate, brine, dried (MgSO₄), filtered and evaporated in vacuo. The obtained residue was purified by column chromatography (5% EtOAc in heptane to 60% EtOAc in heptane) to afford title compound 6 (3.55 g, 96% yield) as a pale green solid: mp 121-122° C.

### Synthesis methyl 9H-carbazole-3-carboxylate (compound 18)

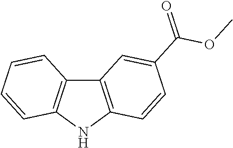 [SMILES: COC(=O)c1ccc2[nH]c3ccccc3c2c1]

In a 100 mL round-bottom flask, a mixture of palladium acetate (1,821 g, 8.11 mmol) and diphenylamine 17 (1.676 g, 7.37 mmol) in glacial acetic acid (40 mL) was stirred under reflux for 1 hr. The organic solvent was removed by distillation. The precipitated metallic palladium was separated by transferring the obtained black residue into a folded paper filter and continuous extraction with acetone in a Soxhlet extractor until the condensing solvent turned colorless. The extract was concentrated in vacuo, and the resultant solid was sonicated for 10 min. in a bath sonicator with 1 M hydrochloric acid (100 mL), filtered, rinsed with distilled water (50 mL×3), and then dried in vacuo. The dry precipitate was sublimed under vacuum to afford the title compound as a light yellow solid. Yield: 1.081 g (65%); mp 180-181° C.

### Synthesis of 9H-carbazole-3-carboxylic acid (compound 19)

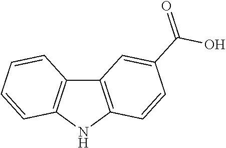 [SMILES: O=C(O)c1ccc2[nH]c3ccccc3c2c1]

Potassium hydroxide (3 g, 53.47 mmol) was added to a stirred solution of 9H-carbazole-3-carboxylic acid methyl ester 18 (818 mg, 3.63 mmol) in a mixture of ethanol (40 mL) and water (10 mL). The reaction mixture was stirred at reflux for 16 hrs and then cooled to room temperature. The solvents were evaporated under reduced pressure, and the residue was diluted with DI water. The solution was placed in an ice-water bath, and acidified to pH ca. 2 by dropwise addition of 1M aqueous HCl. The precipitated product was extracted with ethyl acetate, washed with brine under acidic pH, and dried over MgSO₄. After evaporation of the solvent under reduced pressure, the residue was chromatographed on silica gel with 70% ethyl acetate in heptane to yield the title compound as a beige solid. Yield: 737 mg (96%); mp 271-272° C.

### Synthesis of (9H-carbazol-3-yl)(piperidin-1-yl)methanone (compound 21)

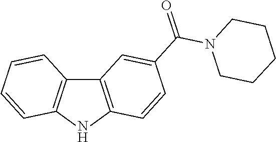 [SMILES: O=C(c1ccc2[nH]c3ccccc3c2c1)N1CCCCC1]

Using 9H-carbazole-3-carboxylic acid 19 (742 mg, 3.51 mmol) and piperidine (416 μL, 4.21 mmol) as starting compounds, the title compound was prepared following the procedures described in preparation of Compound 4 as a beige solid. Yield: 839 mg (86%); mp 221-222° C.

### Synthesis of (9-(3-methoxypropyl)-9H-carbazol-3-yl)(piperidin-1-yl)methanone (compound 22)

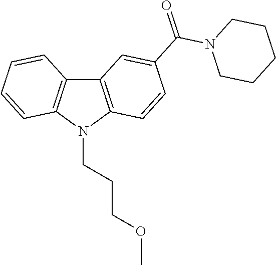 [SMILES: COCCCn1c2ccccc2c2cc(C(=O)N3CCCCC3)ccc21]

Under argon atmosphere, a solution of carbazole 1 (100 mg, 0.36 mmol), 1-bromo-3-methoxypropane (61 μL, 0.54 mmol), and Cs₂CO₃ (234 mg, 0.72 mmol) in DMF (10 mL) was tightly capped in a 25 mL microwave vessel. The mixture was subjected to microwave irradiation at 140° C. for 1 h and then cooled to room temperature. The reaction mixture was diluted with ethyl acetate and filtered. The organic solvents were evaporated in vacuo. The residue was suspended in methyl tert-butyl ether (150 mL), and the organic phase was washed with saturated aqueous sodium bicarbonate, brine, dried (MgSO₄), filtered and evaporated in mow. The obtained residue was purified by column chromatography on silica gel eluting with ethyl acetate/heptanes in different proportions to give 4 (119 mg, 95%) as a clear viscous syrup.

### Example 14: Synthesis of piperidin-1-yl(9-((tetrahydro-2H-pyran-4-yl)methyl)-9H-carbazol-3-yl)methanone (compound 23)

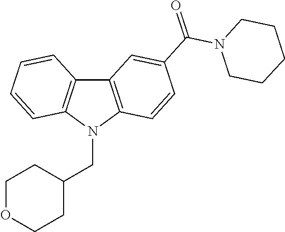 [SMILES: O=C(c1ccc2c(c1)c1ccccc1n2CC1CCOCC1)N1CCCCC1]

Using carbazole amide 21 (100 mg, 0.36 mmol) and 4-(bromomethyl)tetrahydro-2H-pyran (97 mg, 0.54 mmol) as starting compounds, the title compound was prepared following the procedures described in preparation of compound 22 as a colorless foam. Yield: 120 mg (89%).

### Example 15: Synthesis of methyl 4-(3-(piperidine-1-carbonyl)-9H-carbazol-9-yl)butanoate (compound 24)

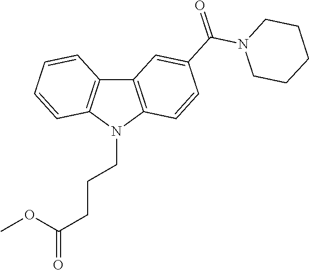 [SMILES: COC(=O)CCCn1c2ccccc2c2cc(C(=O)N3CCCCC3)ccc21]

Using carbazole amide 21 (100 mg, 0.36 mmol) and methyl 4-bromobutanoate (54 μL, 0.43 mmol) as starting compounds, the title compound was prepared following the procedures described in preparation of compound 22 as a colorless glass. Yield: 127 mg (93%).

### Example 16: Synthesis of (9-(3-(dimethylamino)propyl)-9H-carbazol-3-yl)(piperidin-1-yl)methanone (compound 25)

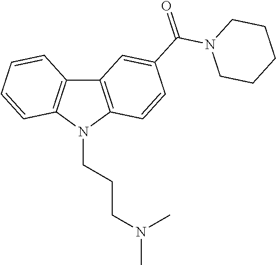 [SMILES: CN(C)CCCn1c2ccccc2c2cc(C(=O)N3CCCCC3)ccc21]

Under argon atmosphere, a solution of carbazole amide 21 (248 mg, 0.89 mmol), 3-chloropropyldimethylamine hydrochloride (311 mg, 1.97 mmol), TBAI (128 mg, 0.35 mmol), and Cs₂CO₃ (886 mg, 2.72 mmol) in DMF (10 mL) was tightly capped in a 25 mL microwave vessel. The mixture was subjected to microwave irradiation at 140° C. for 2 h and then cooled to room temperature. The reaction mixture was diluted with ethyl acetate (50 mL) and filtered. The organic solvents were evaporated in vacuo. The residue was suspended in methyl tert-butyl ether (150 mL), and the organic phase was washed with saturated aqueous sodium bicarbonate, brine, dried (MgSO₄), filtered and evaporated in vacuo. The obtained residue was purified by column chromatography on a BIOTAGE KP-NH (amino-modified silica gel) cartridge using heptanes/ethyl acetate in different proportions to afford the title compound as a clear viscous oil. Yield: 180 mg, 56%.

### Example 17: piperidin-1-yl(9-(pyridin-2-ylmethyl)-9H-carbazol-3-yl)methanone (compound 26)

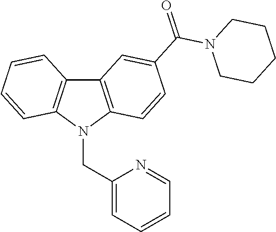 [SMILES: O=C(c1ccc2c(c1)c1ccccc1n2Cc1ccccn1)N1CCCCC1]

(9H-carbazol-3-yl)(piperidin-1-yl)methanone 21 (100 mg, 0.36 mmol) and potassium tert-butoxide (305 mg, 2.72 mmol) were added to DMF (5 mL) in a 25 mL round bottom flask. 2-(Bromomethyl)pyridine hydrobromide (340 mg, 1.34 mmol) was added to the reaction mixture in one portion. The reaction mixture was stirred at room temperature for 2 days, diluted with ethyl acetate (25 mL) and filtered. The solvents were evaporated in vacuo, and the obtained syrup was extracted into ethyl acetate. The organic layer was washed with an aqueous sodium bicarbonate, brine, dried (MgSO₄) and filtered. The volatiles were removed in vacuo, and the obtained syrup was purified by silica gel chromatography using EtOAc/heptane solvent gradient on a BIOTAGE® KP-NH cartridge to afford the target product as a yellowish foam. Yield 106 mg (80%).

### Example 18: Synthesis of piperidin-1-yl(9-(pyridin-4-ylmethyl)-9H-carbazol-3-yl)methanone (compound 27)

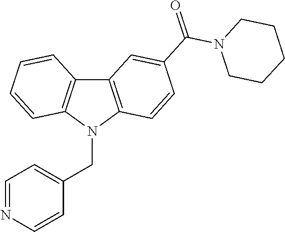 [SMILES: O=C(c1ccc2c(c1)c1ccccc1n2Cc1ccncc1)N1CCCCC1]

Using carbazole amide 21 (100 mg, 0.36 mmol) and 4-(bromomethyl)pyridine hydrobromide (136 mg, 0.54 mmol) as starting compounds, the title compound was prepared following the procedures described in preparation of compound 26 as a yellowish foam. Yield: 118 mg (89%).

### Example 19: Synthesis of piperidin-1-yl(9-(pyridin-3-ylmethyl)-9H-carbazol-3-yl)methanone (compound 28)

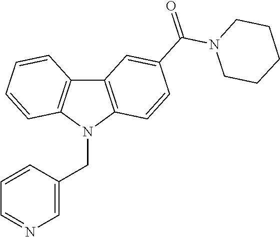 [SMILES: O=C(c1ccc2c(c1)c1ccccc1n2Cc1cccnc1)N1CCCCC1]

Using carbazole amide 21 (100 mg, 0.36 mmol) and 3-(bromomethyl)pyridine hydrobromide (136 mg, 0.54 mmol) as starting compounds, the title compound was prepared following the procedures described in preparation of compound 26 as a yellowish foam. Yield: 45 mg (34%).

### Example 20: Synthesis of (7-methoxy-9-pentyl-9H-carbazol-3-yl)(piperidin-1-yl)methanone (compound 33)

### Synthesis of methyl 4-(3-methoxyphenylamino)benzoate (compound 29)

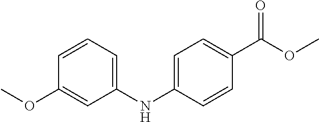

Under argon atmosphere, a solution of methyl 4-bromobenzoate (3.5 g, 16.28 mmol), 3-methoxyaniline (2 g, 16.24 mmol), palladium (II) acetate (218 mg, 0.97 mmol), rac-BINAP (506 mg, 0.81 mmol), and potassium carbonate (6.72 g, 48.62 mmol) in toluene (ca. 10 mL) was tightly capped in a 25 mL microwave vessel. The mixture was subjected to microwave irradiation at 160° C. for 2 h and then cooled to room temperature. The reaction mixture was allowed to cool down to room temperature, diluted with CH₂Cl₂ (50 mL) and filtered. The solvents were removed under reduced pressure, and the obtained residue was distilled in vacuo to afford a yellowish oil: b.p. 160-165° C. at 0.2 mm Hg. Yield: 3.348 g (80%).

### Synthesis of methyl 7-methoxy-9H-carbazole-3-carboxylate (compound 30)

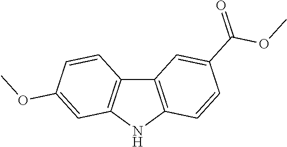

Using benzoate 29 (1.897 g, 7.37 mmol) and palladium acetate (1.987 g, 8.85 mmol) as starting compounds, the title compound was prepared following the procedures described in preparation of compound 18 as light yellow crystals. Yield: 1.6 g (85%).

### Synthesis of methyl 7-methoxy-9-pentyl-9H-carbazole-3-carboxylate (compound 31)

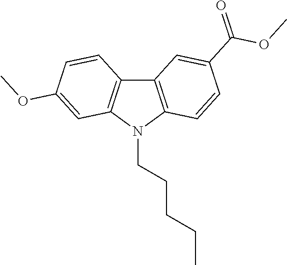

Under argon atmosphere, a solution of carbazole 30 (462 m 81 mmol), 1-bromopentane (320 μL, 2.59 mmol), and Cs₂CO₃ (1.123 g, 3.45 mmol) in DMF (10 mL) was tightly capped in a 25 mL microwave vessel. The mixture was subjected to microwave irradiation at 140° C. for 2 h and then cooled to room temperature. The reaction mixture was diluted with ethyl acetate and filtered. The organic solvents were evaporated in vacuo. The residue was suspended in methyl tert-butyl ether (150 mL), and the organic phase was washed with saturated aqueous sodium bicarbonate, brine, dried (MgSO₄), filtered and evaporated in vacuo. The obtained residue was purified by column chromatography on silica gel, eluent: EtOAc-heptanes (1/99, v/v)→EtOAc-heptanes (2/3, v/v) to give the title compound (480 mg, 82%) as a pale yellow viscous oil.

### Synthesis 7-methoxy-9-pentyl-9H-carbazole-3-carboxylic acid (compound 32): “ester hydrolysis” approach

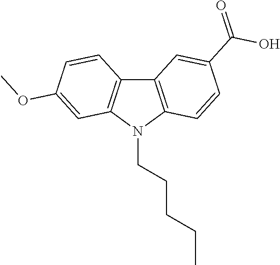 [SMILES: CCCCCn1c2ccc(C(=O)O)cc2c2ccc(OC)cc21]

Potassium hydroxide (3 g, 53.47 mmol) was added to a stirred solution of methyl ester 31 (626 mg, 1.92 mmol) in a mixture of ethanol (40 mL) and water (10 mL). The reaction mixture was stirred at reflux for 16 hrs and then cooled to room temperature. The solvents were evaporated under reduced pressure, and the residue was diluted with DI water. The solution was placed in an ice-water bath, and acidified to pH ca. 2 by dropwise addition of 1M aqueous HCl. The precipitated product was extracted with ethyl acetate, washed with brine under acidic pH (ca. 2), and dried over MgSO₄. After evaporation of the solvent under reduced pressure, the residue was chromatographed on silica gel with 70% ethyl acetate in heptane to yield the title compound as a beige solid. Yield: 480 mg (80%).

### Synthesis of (7-methoxy-9-pentyl-9H-carbazol-3-yl)(piperidin-1-yl)methanone (compound 33)

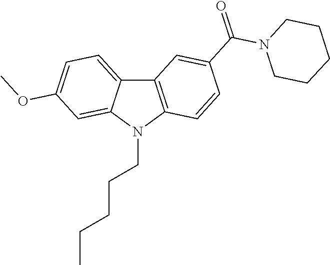 [SMILES: CCCCCn1c2ccc(C(=O)N3CCCCC3)cc2c2ccc(OC)cc21]

9H-carbazole-3-carboxylic acid 32 (1000 mg, 3.21 mmol), piperidine (636 μL, 6.42 mmol), DIPEA (1100 μL, 6.42 mmol), and DMAP (785 mg, 6.42 mmol) were added to DCM (100 mL) under nitrogen. The obtained solution was cooled down on an ice-water bath. EDC (1231 mg, 6.42 mmol) was added to the solution, and the reaction mixture was then allowed to warm to room temperature and stirred for 16 h. The solvent was removed in vacuo, and the obtained residue was extracted into ethyl acetate (150 mL). The organic layer was washed consecutively with 5% citric acid solution (50 mL×3), concentrated sodium bicarbonate (50 mL 3), brine (50 mL), dried over magnesium sulfate, filtered, and concentrated in vacuo. The residue was purified on silica gel using heptanes/ethyl acetate in different proportions to afford the title compound as a yellowish oil (1109 mg, 91%).

### Example 21: Synthesis of N,N-diethyl-7-methoxy-9-pentyl-9H-carbazole-3-carboxamide (compound 34)

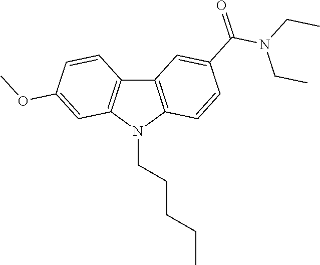 [SMILES: CCCCCn1c2ccc(C(=O)N(CC)CC)cc2c2ccc(OC)cc21]

Using 9H-carbazole-3-carboxylic acid 32 (112 mg, 0.36 mmol) and diethylamine (40 μL, 0.38 mmol) as starting compounds, the title compound was prepared following the procedures described in preparation of compound 33 as a colorless viscous oil. Yield: 126 mg (96%).

### Example 22: Synthesis of 7-methoxy-9-pentyl-N-(piperidin-1-yl)-9H-carbazole-3-carboxamide (compound 35)

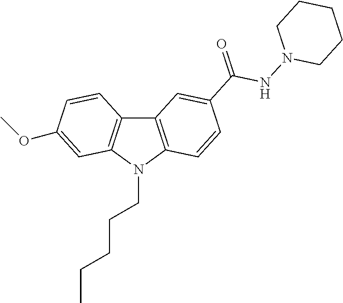 [SMILES: CCCCCn1c2ccc(C(=O)NN3CCCCC3)cc2c2ccc(OC)cc21]

Using 9H-carbazole-3-carboxylic acid 32 (122 mg, 0.39 mmol) and piperidin-1-amine (64 mg, 0.64 mmol) as starting compounds, the title compound was prepared following the procedures described in preparation of compound 33 as a white solid. Yield: 56 mg (36%); mp 178-179° C.

### Example 23: Synthesis of 7-methoxy-9-pentyl-9H-carbazol-3-yl)(morpholino)methanone (compound 36)

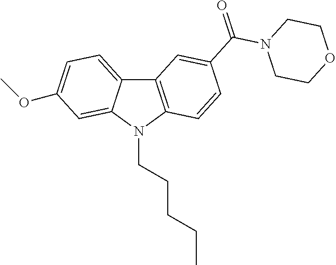 [SMILES: CCCCCn1c2ccc(C(=O)N3CCOCC3)cc2c2ccc(OC)cc21]

Using 9H-carbazole-3-carboxylic acid 32 (100 mg, 0.32 mmol) and morpholine (56 mg, 0.64 mmol) as starting compounds, the title compound was prepared following the procedures described in preparation of compound 33 as a yellow glass. Yield: 94 mg (77%).

### Example 24: Synthesis of (7-methoxy-9-pentyl-9H-carbazol-3-yl)(4-methylpiperazin-1-yl)methanone (compound 37)

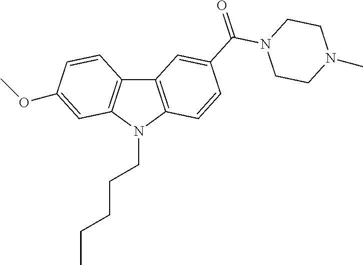

Using 9H-carbazole-3-carboxylic acid 32 (118 mg, 0.38 mmol) and 1-methylpiperazine (64 mg, 0.64 mmol) as starting compounds, the title compound was prepared following the procedures described in preparation of compound 33 as an orange viscous oil. Yield: 132 mg (89%).

### Example 25: Synthesis of N-(adamantan-1-yl)-7-methoxy-9-pentyl-9H-carbazole-3-carboxamide (compound 38)

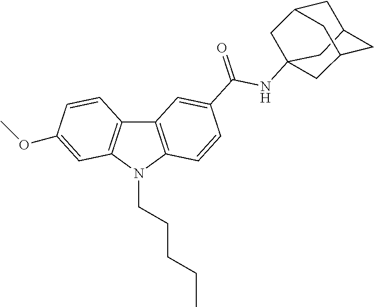

Using 9H-carbazole-3-carboxylic acid 32 (118 mg, 0.38 mmol) and 1-adamantylamine (63 mg, 0.41 mmol) as starting compounds, the title compound was prepared following the procedures described in preparation of compound 33 as a white solid. Yield: 163 mg (97%); mp 141-142° C.

### Example 26: Synthesis of 9-pentyl-6-[(piperidin-1-yl)carbonyl]-9H-carbazol-2-ol (compound 39)

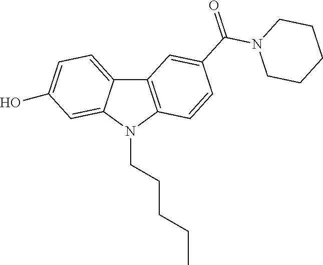 [SMILES: CCCCCn1c2ccc(C(=O)N3CCCCC3)cc2c2ccc(O)cc21]

Under argon atmosphere, a solution of carbazole 1 (100 mg, 0.26 mmol) and sodium ethanethiolate (200 mg, 2.38 mmol) in DMF (5 mL) was tightly capped in a 25 mL microwave vessel. The mixture was subjected to microwave irradiation at 120° C. for 9 h and then cooled to room temperature. The organic solvent was evaporated in vacuo, and the residue was purified by column chromatography on silica gel using heptanes/ethyl acetate in different proportions to yield carbazole 39 as white crystals. Yield: 71 mg (61%); mp 197-198° C.

### Example 27: Synthesis of 2-[(ethylsulfanyl)methoxy]-9-pentyl-6-[(piperidin-1-yl)carbonyl]-9H-carbazole (compound 40)

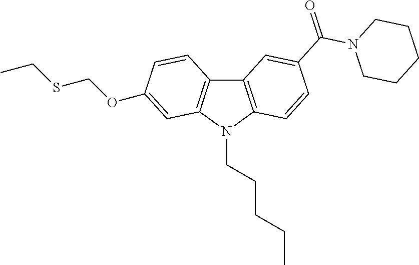 [SMILES: CCCCCn1c2ccc(C(=O)N3CCCCC3)cc2c2ccc(OCSCC)cc21]

The title compound was purified as from the synthesis of carbazole 39. Yield: 18 mg (16%); clear glass.

### Example 28: Synthesis of 9-(cyclohexylmethyl)-2-methoxy-6-[(piperidin-1-yl)carbonyl]-9H-carbazole (compound 44)

### Synthesis of 9-(cyclohexylmethyl)-2-methoxy-6-methyl-9H-carbazole (compound 42)

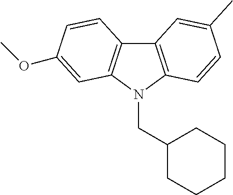

Under argon atmosphere, a solution of 7-methoxy-3-methylcarbazole (Krahl et al., Organic & Biomolecular Chemistry 4:3215 (2006)) (600 mg, 2.84 mmol), bromomethylcyclohexane (635 μL, 4.55 mmol), and Cs₂CO₃ (1.8 g) in DMF (15 mL) was subjected to microwave irradiation at 140° C. for 1 h. The reaction mixture was cooled, diluted with ethyl acetate (150 mL), filtered, and extracted with diethyl ether. The organic layer was washed consecutively with concentrated sodium bicarbonate (150 mL×3), brine, dried over magnesium sulfate, filtered, and concentrated in vacuo. The obtained residue was purified by column chromatography on silica gel using heptanes/ethyl acetate in different proportions to afford the title compound as a brownish solid. Yield: 743 mg (85%), mp 136-137° C.

### Synthesis of 9-(cyclohexylmethyl)-7-methoxy-9H-carbazole-3-carboxylic acid (compound 43)

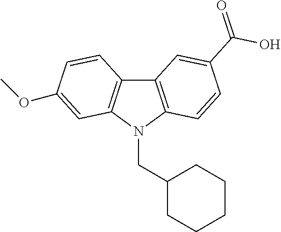 [SMILES: COc1ccc2c3cc(C(=O)O)ccc3n(CC3CCCCC3)c2c1]

Potassium permanganate (5157 mg, 32.63 mmol) was added portionwise over a period of 5 h to a stirred solution of carbazole 42 (743 mg, 2.42 mmol) in water/tert-butanol (160 mL, 1:2, v/v) on a bath heated to 100° C. The mixture was then quenched with ethanol (25 mL) and allowed to cool to room temperature. The reaction mixture was filtered through a pad of celite and concentrated in vacuo to remove organic solvent. The obtained solution was diluted with water (250 mL), basified with NaOH to pH ca. 10, and extracted with diethyl ether to remove the unreacted starting material. The aqueous solution was cooled on an ice-water bath and acidified with ice-cold solution of sulfuric acid (20%) to pH ca. 2. The resultant bulky precipitate was extracted into ethyl acetate and the extract was washed with brine, dried over magnesium sulfate, filtered, and concentrated in vacuo. The precipitated product was collected by filtration, washed several times with heptanes, and dried overnight to produce the title compound as a beige solid. Yield: 235 mg (29%).

### Synthesis of 9-(cyclohexylmethyl)-2-methoxy-6-[(piperidin-1-yl)carbonyl]-9H-carbazole (compound 44)

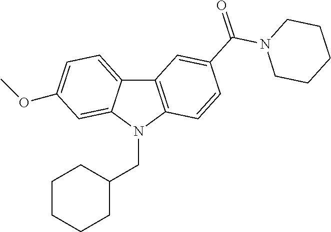 [SMILES: COc1ccc2c3cc(C(=O)N4CCCCC4)ccc3n(CC3CCCCC3)c2c1]

9H-carbazole-3-carboxylic acid 43 (153 mg, 0.45 mmol), piperidine (90 μL, 0.90 mmol), DIPEA (154 μL, 0.90 mmol), and DMAP (111 mg, 0.91 mmol) were added to DCM (30 mL) under nitrogen. The obtained solution was cooled down on an ice-water bath. EDC (174 mg, 0.90 mmol) was added to the solution, and the reaction mixture was then allowed to warm to room temperature and stirred for 16 h. The solvent was removed in vacuo, and the obtained residue was extracted into ethyl acetate (150 mL). The organic layer was washed consecutively with 5% citric acid solution (50 mL×3), concentrated sodium bicarbonate (50 mL×3), brine (50 mL), dried over magnesium sulfate, filtered, and concentrated in vacuo. The residue was purified on silica gel using heptanes/ethyl acetate in different proportions to afford the title compound as a white solid. Yield: 163 mg (89%), mp 91-92° C.

### Example 29: Synthesis of N-(4-chlorophenethyl)-9-(cyclohexylmethyl)-7-methoxy-9H-carbazole-3-carboxamide (compound 45)

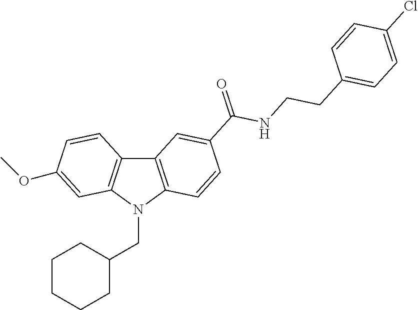 [SMILES: COc1ccc2c3cc(C(=O)NCCc4ccc(Cl)cc4)ccc3n(CC3CCCCC3)c2c1]

Using 9H-carbazole-3-carboxylic acid 43 (80 mg, 0.24 mmol) and 2-(4-chlorophenyl)ethanamine (74 mg, 0.48 mmol) as starting compounds, the title compound was prepared following the procedures described in preparation of compound 44 as a white solid. Yield: 58 mg (51%); mp 179-181° C.

### Example 30: Synthesis of (7-methoxy-9-pentyl-9H-carbazol-3-yl)(piperidin-1-yl)methanethione (compound 46)

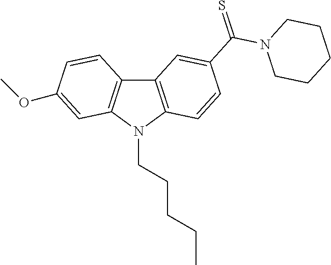 [SMILES: CCCCCn1c2ccc(C(=S)N3CCCCC3)cc2c2ccc(OC)cc21]

Using carbazole amide 33 (60 mg, 0.16 mmol) and Lawesson's reagent (106 mg, 0.26 mmol) as starting compounds, the title compound was prepared following the procedures described in preparation of compound 13 as a white solid. Yield: 57 mg (91%); mp 116-118° C.

### Example 31: Synthesis of 2-methoxy-9-pentyl-6-(piperidin-1-ylmethyl)-9H-carbazole (compound 47)

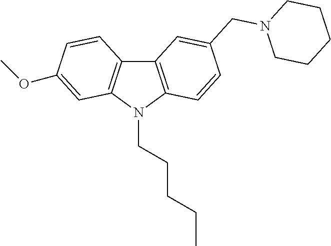 [SMILES: CCCCCn1c2ccc(CN3CCCCC3)cc2c2ccc(OC)cc21]

Lithium aluminium hydride (187 mg, 4.98 mmol) was dissolved in dry THF (30 mL) in a 100 mL flask equipped with a rubber syringe cap and a magnetic stirring bar. The obtained solution was cooled to 0° C. under argon by immersing the flask into an ice-water bath. Carbazole 33 (87 mg, 0.23 mmol) was dissolved in dry THF (4 mL) under argon, and the obtained solution was added dropwise to the solution of LAH via a hypodermic needle of a 5 mL plastic syringe with vigorous stirring over a period of 3 min. The temperature was maintained approximately at 0° C. during the addition. The resulting mixture was stirred at 0° C. for 1 h, and then quenched by successive dropwise addition of 5% sodium dithionite solution (ca. 3 mL). The resulting suspension was centrifuged, and the supernatant liquid was extracted with diethyl ether (150 mL). The ether extract was washed with 8 M NaOH solution, brine, dried over Mg₂SO₄, filtered and then evaporated on a rotary evaporator. The obtained residue was purified by column chromatography using a BIOTAGE® KP-NH cartridge to give the target compound as a colorless oil which crystallized on standing. Yield: 78 mg (93%); mp 64-65° C.

### Example 32: Synthesis of 2-(methylsulfanyl)-9-pentyl-6-[(piperidin-1-yl)carbonyl]-9H-carbazole (compound 52)

### Synthesis of methyl 4-(3-(methylthio)phenylamino)benzoate compound 48)

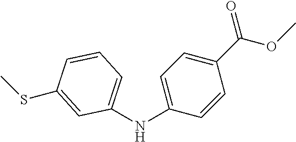

Using 4-bromobenzoate (3500 mg, 16.28 mmol) and aniline (2266 mg, 16.28 mmol) as starting compounds, the title compound was prepared following the procedures described in preparation of Compound 17 as a brownish oil. Yield: 2879 mg (65%).

### Synthesis of methyl 7-(methylthio)-9H-carbazole-3-carboxylate (compound 49)

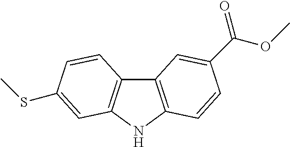

A mixture of diarylamine (1594 mg, 5.83 mmol), Pd(OAc)₂ (1309 mg, 5.83 mmol) and Cu(OAc)₂ (2118 mg, 11.66 mmol) in glacial acetic acid (10 mL) was introduced in a 25 mL microwave vessel and irradiated at 160° C. for 1 h. After completion of the reaction, the reaction mixture was cooled to room temperature, diluted with ethyl acetate (50 mL) and filtered. The organic solvents were removed in vacuo, and the obtained residue was purified by silica gel column chromatography eluting with heptanes/ethyl acetate in different proportions to afford the title compound as a beige solid. Yield: 790 mg (50%).

### Synthesis of methyl 7-(methylthio)-9-pentyl-9H-carbazole-3-carboxylate (compound 50)

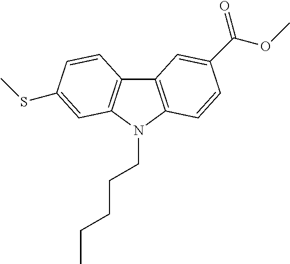

Using thiocarbazole 49 (245 mg, 0.90 mmol), 1-bromopentane (170 μL, 1.36 mmol) as starting compounds, the title compound was prepared following the procedures described in preparation of compound 31 as a yellow oil. Yield: 307 mg (100%).

### Synthesis of 7-(methylthio)-9-pentyl-9H-carbazole-3-carboxylic acid (compound 51)

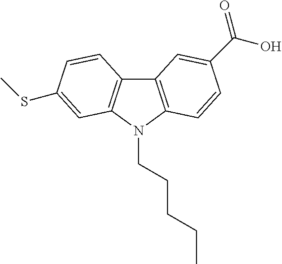 [SMILES: CCCCCn1c2ccc(C(=O)O)cc2c2ccc(SC)cc21]

Using methyl ester 50 (307 mg, 0.90 mmol) and potassium hydroxide (2 g, 35.71 mmol), the title compound was prepared following the procedures described in preparation of compound 32 as a beige solid. Yield: 260 mg (88%); mp 211-212° C.

### Synthesis of 2-(methylsulfanyl)-9-pentyl-6-[(piperidin-1-yl)carbonyl]-9H-carbazole (compound 52)

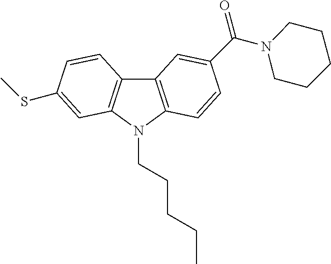 [SMILES: CCCCCn1c2ccc(C(=O)N3CCCCC3)cc2c2ccc(SC)cc21]

Using carboxylic acid 51 (195 mg, 0.60 mmol) and piperidine (118 μL, 1.19 mmol), as starting compounds, the title compound was prepared following the procedures described in preparation of compound 33 as an off-white solid. Yield: 195 mg (83%); 140-141° C.

### Example 33: Synthesis of 2-methanesulfonyl-9-pentyl-6-[(piperidin-1-yl)carbonyl]-9H-carbazole (compound 53)

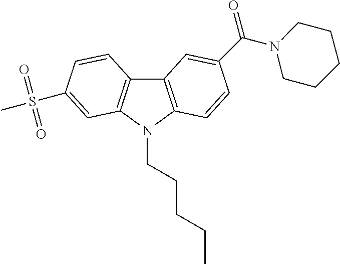 [SMILES: CCCCCn1c2ccc(C(=O)N3CCCCC3)cc2c2ccc(S(C)(=O)=O)cc21]

To a solution of amide 52 (114 mg, 0.29 mmol) in 30 mL of methylene chloride was added freshly purified m-chloroperoxybenzoic acid (150 mg, 0.87 mmol). The resulting solution was stirred at room temperature for 1 h and then diluted with 1 N solution of NaOH (5 mL). The reaction mixture was extracted with methylene chloride (30 mL×3). The combined organic layers were dried over Mg₂SO₄ and filtered. The organic solvent was removed in vacuo, and the crude product was purified by silica gel chromatography to give the target compound as a yellowish glass (129 mg, 79%).

### Example 34: 1,1-dimethyl-4-[(9-pentyl-9H-carbazol-3-yl)carbonyl]piperazin-1-ium iodide (compound 54)

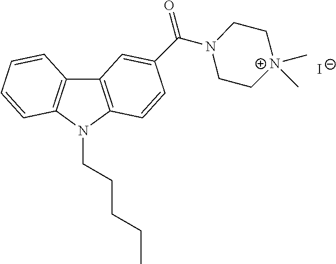 [SMILES: CCCCCn1c2ccccc2c2cc(C(=O)N3CC[N+](C)(C)CC3)ccc21 ; [I-]]

Methyl iodide (764 μL, 12.32 mmol) was added to a stirred solution of amine 9 (320 mg, 0.88 mmol) in anhydrous diethyl ether (10 mL). A precipitate immediately started forming, and stirring was continued for 18 h at room temperature. The precipitated solid was isolated by filtration, washed with diethyl ether (ca. 100 mL), and dried under vacuum to provide the title compound (186 mg, 42%) as an off-white powder: mp 119-120° C. with decomposition.

### Example 35: Synthesis of 4-[(7-methoxy-9-pentyl-9H-carbazol-3-yl)carbonyl]-1,1-dimethylpiperazin-1-ium iodide (compound 55)

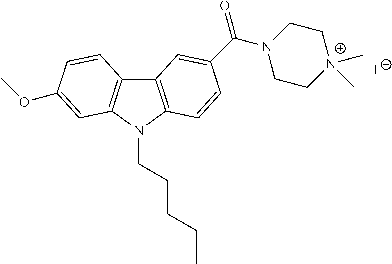

Using amine 37 (259 mg, 0.66 mmol) and methyl iodide (764 μL, 12.32 mmol), as starting compounds, the title compound was prepared following the procedures described in preparation of compound 54 as an off-white solid. Yield: 156 mg (44%); mp 145-147° C. with decomposition.

### Example 36: Synthesis of 1-({9-pentyl-9H-pyrido[3,4-b]indol-3-yl}carbonyl)piperidine (compound 58)

### Synthesis of ethyl 9-pentyl-9H-pyrido[3,4-b]indole-3-carboxylate (56)

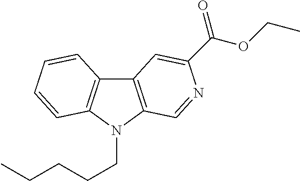 [SMILES: CCCCCn1c2ccccc2c2cc(C(=O)OCC)ncc21]

Using ethyl 9H-pyrido[3,4-b]indole-3-carboxylate (1000 mg, 4.16 mmol) and n-bromopentane (772 □L, 6.24 mmol), as starting compounds, the title compound was prepared following the procedures described in preparation of compound 31 as a white solid. Yield: 1082 mg (84%), mp 92-93° C.

### Synthesis of 9-pentyl-9H-pyrido[3,4-b]indole-3-carboxylic acid (57)

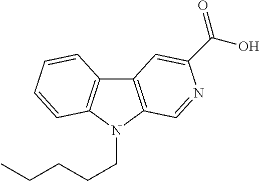 [SMILES: CCCCCn1c2ccccc2c2cc(C(=O)O)ncc21]

Using ethyl ester 56 (715 mg, 2.30 mmol) and potassium hydroxide (3000 mg, 53.57 mmol), the title compound was prepared following the procedures described in preparation of compound 32 as a pinkish solid. Yield: 647 mg (100%).

### Synthesis of 1-({9-pentyl-9H-pyrido[3,4-b]indol-3-yl}carbonyl)piperidine (58)

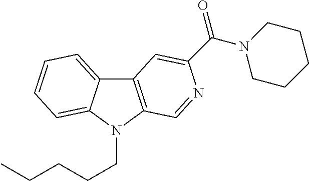 [SMILES: CCCCCn1c2ccccc2c2cc(C(=O)N3CCCCC3)ncc21]

Using acid 57 (241 mg, 0.85 mmol) and piperidine (102 mg, 1.20 mmol) as starting compounds, the tide compound was prepared following the procedures described in preparation of compound 4 as an off-white solid. Yield: 122 mg (41%); mp 126-127° C.

### Example 37: Synthesis of 9-pentyl-N-(piperidin-1-yl)-9H-pyrido[3,4-b]indole-3-carboxamide (59)

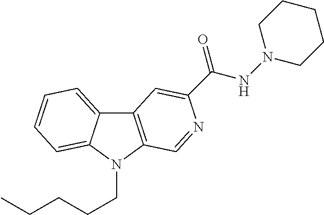 [SMILES: CCCCCn1c2ccccc2c2cc(C(=O)NN3CCCCC3)ncc21]

Using acid 57 (100 mg, 0.35 mmol) and 1-aminopiperidine (36 mg, 0.36 mmol) as starting compounds, the title compound was prepared following the procedures described in preparation of compound 4 as a yellowish glass. Yield: 71 mg (56%).

### Example 38: Synthesis of N-(2,2-dimethylpropyl)-9-pentyl-9H-pyrido[3,4-b]indole-3-carboxamide (60)

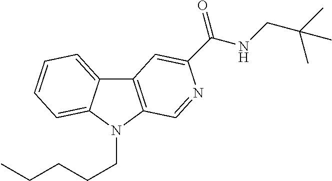 [SMILES: CCCCCn1c2ccccc2c2cc(C(=O)NCC(C)(C)C)ncc21]

Using acid 57 (100 mg, 0.35 mmol) and neopentylamine (37 mg, 0.42 mmol) as starting compounds, the title compound was prepared following the procedures described in preparation of compound 4 as a white solid. Yield: 67 mg (54%); mp 91-92° C.

### Example 39: 2-benzoyl-7-methoxy-5-pentyl-1H,2H,3H,4H,5H-pyrido[4,3-b]indole (compound 64)

### Synthesis of ethyl 7-methoxy-3,4-dihydro-1H-pyrido[4,3-b]indole-2(5H)-carboxylate (compound 61)

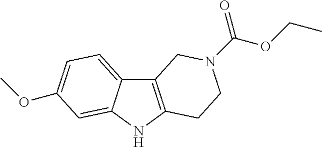 [SMILES: CCOC(=O)N1CCc2[nH]c3cc(OC)ccc3c2C1]

G-carboline 61 was prepared by heating phenylhydrazine hydrochloride (4.750 g, 27.2 mmol) and 1-carbethoxy-4-piperidone (5.588 g, 32.64 mmol) in anhydrous ethanol (150 mL) at reflux for 16 h. The solvent was evaporated in vacuo, and the obtained residue was purified by silica gel chromatography using ethyl acetate/heptanes in different proportions to afford the title compound as a white solid. Yield 4.52 g (61%).

### Synthesis of ethyl 7-methoxy-5-pentyl-3,4-dihydro-1H-pyrido[4,3-b]indole-2(5H)-carboxylate (compound 62)

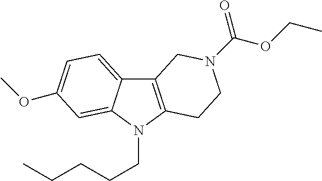 [SMILES: CCCCCn1c2c(c3ccc(OC)cc31)CN(C(=O)OCC)CC2]

Under argon atmosphere, a solution of g-carboline 61 (1 g, 3.65 mmol), n-pentyl bromide (1 mL, 8.06 mmol), and Cs₂CO₃ (2638 mg, 8.10 mmol) in DMF (5 mL) was tightly capped in a 25 mL microwave vessel. The mixture was subjected to microwave irradiation at 140° C. for 2 h and then cooled to room temperature. The reaction mixture was diluted with ethyl acetate and filtered. The organic solvents were evaporated in vacuo. The residue was suspended in ethyl acetate (150 mL), and the organic phase was washed with saturated aqueous sodium bicarbonate, brine, dried (MgSO₄), filtered and evaporated in vacuo. The obtained residue was purified by column chromatography on silica gel using ethyl acetate/heptanes in different proportions to afford the title compound (837 mg, 67%) as a yellowish oil.

### Synthesis of 7-methoxy-5-pentyl-2,3,4,5-tetrahydro-1H-pyrido[4,3-b]indole (compound 63)

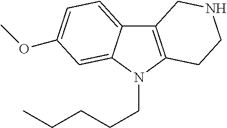 [SMILES: CCCCCn1c2c(c3ccc(OC)cc31)CNCC2]

Solid KOH (3 g, 53.57 mmol) was added to a solution of carbethoxyindole 62 (859 mg, 2.49 mmol) in a mixture of ethanol (80 mL) and water (10 mL). The resulting solution was heated at reflux under N₂ for 48 h. The obtained solution was concentrated in vacuo to remove ethanol, diluted with saturated aqueous sodium bicarbonate (50 mL) and extracted with ethyl acetate (150 mL). The organic phase was washed with saturated aqueous sodium bicarbonate (50 mL×2), brine (50 mL), dried (MgSO₄), filtered and evaporated in vacuo. The obtained residue was purified on a BIOTAGE® KP-NH cartridge (amino-modified silica gel) using heptanes/ethyl acetate in different proportions to afford the title compound as a yellowish viscous oil (635 mg, 93%). In order to convert the free base form of 63 into its hydrochloride salt, the obtained oil was dissolved in ethanol (50 mL) and a 36% solution of HCl (480 mL, 4.73 mmol) was added. The solvent was removed in vacuo, and coevaporation with anhydrous ethanol (50 mL) was repeated twice. The concentrated ethanol solution (ca. 2 mL) was placed into a refrigerator and allowed to cool down to a temperature below 0° C. The precipitated product was collected by filtration, washed with pentane (25 mL) and dried in vacuo overnight to afford a white solid. Yield in the form of a hydrochloride salt: 380 mg (49%); mp 210-211° C.

### Synthesis 2-benzoyl-7-methoxy-5-pentyl-1H,2H,3H,4H,5H-pyrido[4,3-b]indole (compound 64)

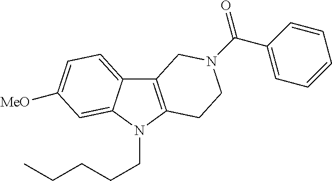 [SMILES: CCCCCn1c2c(c3ccc(OC)cc31)CN(C(=O)c1ccccc1)CC2]

Amine hydrochloride 63 (101 mg, 0.33 mmol) was suspended in anhydrous DCM (30 mL) under nitrogen, and the obtained suspension was cooled with ice-cold water. DIPEA (197.6 mg, 1.18 mmol) was added to the solution, followed by benzoyl chloride (145.2 mg, 0.49 mmol). The flask was removed from the ice bath, and the reaction mixture was stirred for 3 h. After concentration, the residue was purified by column chromatography on silica gel, eluting with EtOAc/heptanes in different proportions to afford 84 mg (68%) of 63 as a pale yellowish solid.

### Example 40: Synthesis of 5-{7-methoxy-5-pentyl-1H,2H,3H,4H,5H-pyrido[4,3-b]indole-2-sulfonyl}-N,N-dimethylnaphthalen-1-amine (compound 65)

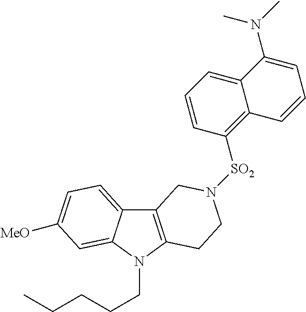 [SMILES: CCCCCn1c2c(c3ccc(OC)cc31)CN(S(=O)(=O)c1cccc3c(N(C)C)cccc13)CC2]

Using amine hydrochloride 63 (100 mg, 0.32 mmol) and solid dansyl chloride (131 mg, 0.49 mmol) as starting compounds, the title compound was prepared following the procedures described in preparation of compound 64 as a pale greenish solid. Yield: 144 mg (88%); mp 71-72° C.

### Example 41: Synthesis of 5-ethyl-7-methoxy-2-[(4-methylnaphthalen-1-yl)carbonyl]-1H,2H,3H,4H,5H-pyrido[4,3-b]indole (Compound 68)

### Synthesis of ethyl 5-ethyl-7-methoxy-3,4-dihydro-1H-pyrido[4,3-b]indole-2(5H)-carboxylate (compound 66)

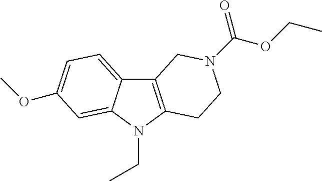 [SMILES: CCOC(=O)N1CCc2c(c3ccc(OC)cc3n2CC)C1]

Sodium hydride (131 mg, 3.28 mmol) in the form of a 60% dispersion in oil was washed with pentanes (25 mL) on a glass filter and added in small portions to a solution of 11.1 (0.5 g, 1.82 mmol) in DMF at 0° C. under N₂. Then, ethyl bromide (203 mL, 2.73 mmol) was added at 0° C. and the mixture was stirred at room temperature for 1 h. The reaction mixture was quenched with saturated aqueous ammonium chloride (3 mL) on an ice-water bath, and extracted with ethyl acetates (150 mL). The organic phase was washed with saturated aqueous sodium bicarbonate (50 mL×2), brine (50 mL), dried (MgSO₄), filtered and evaporated in vacuo. The obtained residue was purified by column chromatography on silica gel using ethyl acetate/heptanes in different proportions to afford the title compound (460 mg, 83%) as a yellowish glass.

### Synthesis of 5-ethyl-7-methoxy-2,3,4,5-tetrahydro-1H-pyrido[4,3-b]indole (compound 67)

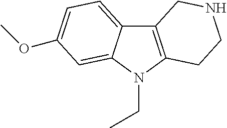 [SMILES: CCn1c2c(c3ccc(OC)cc31)CNCC2]

Using carbethoxyindole 66 (460 mg, 1.69 mmol) and potassium hydroxide (4166 mg, 74.39 mmol), the title compound was prepared following the procedures described in preparation of compound 63 as a yellowish oil (free base form). Yield: 273 mg (70%).

### Synthesis of 5-ethyl-7-methoxy-2-[(4-methylnaphthalen-1-yl)carbonyl]-1H,2H,3H,4H,5H-pyrido[4,3-b]indole (compound 68)

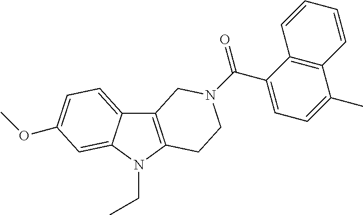

Using free-base form of amine 67 (273 mg, 1.19 mmol) and 4-methyl-1-naphthoyl chloride (364 mg, 1.78 mmol) as starting compounds, the title compound was prepared following the procedures described in preparation of compound 63 as a pale greenish solid. Yield: 261 mg (55%); mp 209-210° C.

### Example 42: (S)-tert-butyl 3-methyl-9-pentyl-3-(piperidine-1-carbonyl),4-dihydro-1H-pyrido[3,4-b]indole-2(9H)-carboxylate (compound 70)

### Synthesis (S)-tert-butyl 3-(piperidine-1-carbonyl)-3,4-dihydro-1H-pyrido[3,4-b]indole-2(9H)-carboxylate (compound 69)

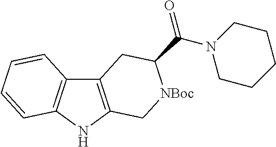 [SMILES: CC(C)(C)OC(=O)N1Cc2[nH]c3ccccc3c2C[C@H]1C(=O)N1CCCCC1]

HOBt (641 mg, 4.74 mmol) was added to the solution of N-Boc-3(L)-1,2,3,4-tetrahydro-b-carboline-3-carboxylic acid (1.0 g, 3.16 mmol) in a mixture of THF (30 mL) and DMF (3 mL) at 0° C. EDC (790 mg, 4.12 mmol) was added to the obtained solution, and stirring was continued for 15 min. A solution of piperidine (624 mL, 6.32 mmol) and DU-A (1.075 mL, 6.32 mmol) in 2 mL of anhydrous THF was added dropwise to the reaction mixture which was stirred at 0° C. for 2 h and then at room temperature for 16 h. The solvent was removed under reduced pressure and the residue was extracted into 100 mL of ethyl acetate and the extract was washed successively with 5% sodium bicarbonate (50 mL×2), 5% citric acid (50 mL×2), and saturated sodium chloride (50 mL). The organic layer was separated, dried over anhydrous magnesium sulfate and filtered. After concentration, the residue was purified by column chromatography on silica gel eluting with EtOAc/heptanes in different proportions to afford 1137 mg (94%) of compound 67 as a white solid.

### Synthesis (S)-tert-butyl 3-methyl-9-pentyl-3-(piperidine-1-carbonyl)-3,4-dihydro-1H-pyrido[3,4-b]indole-2(9H)-carboxylate (compound 70)

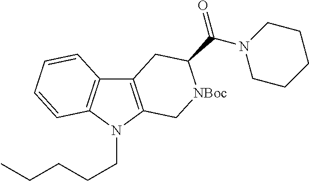 [SMILES: CCCCCn1c2c(c3ccccc31)C[C@@H](C(=O)N1CCCCC1)N(C(=O)OC(C)(C)C)C2]

Under argon atmosphere, a solution of b-carboline amide 69 (1137 mg, 2.96 mmol), 1-bromopentane (836 mL, 6.74 mmol), and Cs₂CO₃ (3663 mg, 11.24 mmol) in DMF (10 mL) was subjected to microwave irradiation at 140° C. for 2 h and then cooled to room temperature. The reaction mixture was diluted with ethyl acetate and filtered. The organic solvents were evaporated in vacuo. The residue was extracted with ethyl acetate (150 mL), and the organic phase was washed with 5% citric acid (50 mL×2), saturated aqueous sodium bicarbonate (50 mL×2), brine (50 dried over MgSO₄, filtered and evaporated in vacuo. The obtained residue was purified by column chromatography on silica gel, eluenting with EtOAc/heptanes in different proportions to give the title compound (637 mg, 47%) as a yellowish glass.

### Example 43: 1-{[(3S)-9-pentyl-1H,2H,3H,4H,9H-pyrido[3,4-b]indol-3-yl]carbonyl}piperidine hydrochloride (compound 71)

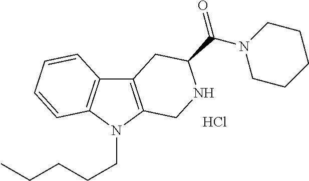 [SMILES: CCCCCn1c2c(c3ccccc31)C[C@@H](C(=O)N1CCCCC1)NC2 ; Cl]

Boc-protected amide 70 (330 mg, 0.73 mmol) was dissolved in DCM (3 mL), and dimethylsulfide (1 mL, 13.52 mmol) and ethanedithiol (100 μL, 1.19 mmol) were added. The solution was cooled to 0° C., and TFA (3 mL, 39.18 mmol) was added. The reaction mixture was stirred at room temperature for 2 h and quenched with water (3 mL). Saturated NaHCO₃ solution was added until pH 7. Then 2N NaOH was added until the solution turned basic (pH ca. 9). The aqueous layer was extracted with ethyl acetate (3×50 mL). The combined organic layers were washed with brine (50 mL), dried over magnesium sulfate, and concentrated in vacuo. The crude product was purified by reversed-phase silica gel chromatography using acetonitrile/water in different proportions to give a colorless oil. The resulting oil was dissolved in 50 mL of anhydrous ethanol, and 36% hydrochloric acid (390 mL) was added. The solvents were evaporated in vacuo, and then co-evaporation with 50 mL of anhydrous ethanol was repeated twice. The obtained concentrated solution (ca. 1 mL) was allowed to cool down in a refrigerator; the precipitated white solid was collected by filtration, rinsed with pentane and dried in vacuo overnight. Yield: 106 mg (37%); mp 227-228° C.

### Example 44: Synthesis of (R)-tert-butyl 3-methyl-9-pentyl-3-(piperidine-1-carbonyl)-3,4-dihydro-1H-pyrido[3,4-b]indole-2(9H)-carboxylate (compound 73)

### Synthesis of (R)-tert-butyl 3-methyl-3-(piperidine-1-carbonyl)-3,4-dihydro-1H-pyrido[3,4-b]indole-2(9H)-carboxylate (compound 72)

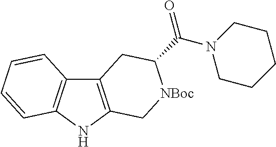 [SMILES: CC(C)(C)OC(=O)N1Cc2[nH]c3ccccc3c2C[C@@H]1C(=O)N1CCCCC1]

Using N-Boc-3(D)-1,2,3,4-tetrahydro-□-carboline-3-carboxylic acid (1.0 g, 3.16 mmol) and piperidine (624 mL, 6.32 mmol) as starting compounds, the title compound was prepared following the procedures described in preparation of compound 69 as a white solid. Yield: 1291 mg (>100%).

### Synthesis of (R)-tert-butyl 3-methyl-9-pentyl-3-(piperidine-1-carbonyl)-3,4-dihydro-1H-pyrido[3,4-b]indole-2(9H)-carboxylate (compound 73)

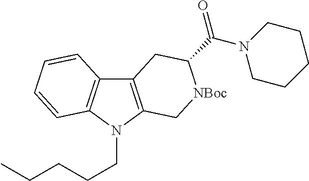 [SMILES: CCCCCn1c2c(c3ccccc31)C[C@H](C(=O)N1CCCCC1)N(C(=O)OC(C)(C)C)C2]

Using b-carboline amide 72 (816 mg, 2.13 mmol) and 1-bromopentane (1 mL, 8.06 mmol) as starting compounds, the title compound was prepared following the procedures described in preparation of compound 70 as a yellowish glass. Yield: 349 mg (36%).

### Example 45: Synthesis of 1-{[(3R)-9-pentyl-1H,2H,3H,4H,9H-pyrido[3,4-b]indol-3-yl]carbonyl}piperidine hydrochloride (compound 74)

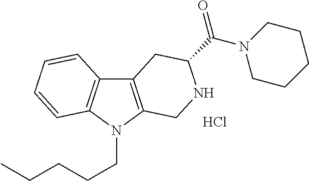 [SMILES: CCCCCn1c2c(c3ccccc31)C[C@H](C(=O)N1CCCCC1)NC2 ; Cl]

Using Boc-protected amide 73 (349 mg, 0.77 mmol), the title compound was prepared following the procedures described in preparation of compound 71 as a white solid. Yield: 162 mg (60%).

### Example 46: Synthesis of 1-{[(3R)-2,9-dipentyl-1H,2H,3H,4H,9H-pyrido[3,4-b]indol-3-yl]carbonyl}piperidine hydrochloride (compound 75)

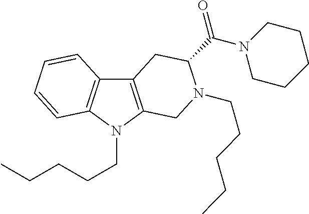 [SMILES: CCCCCN1Cc2c(c3ccccc3n2CCCCC)C[C@@H]1C(=O)N1CCCCC1]

Under argon atmosphere, a solution of b-carboline amide 73 (1200 mg, 3.12 mmol), 1-bromopentane (3.5 mL, 28.22 mmol), and Cs₂CO₃ (4303 mg, 13.20 mmol) in DMF (10 mL) was heated at 140° C. for 19 h and then cooled to room temperature. The reaction mixture was diluted with ethyl acetate and filtered. The organic solvents were evaporated in vacuo. The residue was extracted with ethyl acetate (150 mL), and the organic phase was washed with saturated aqueous sodium bicarbonate (50 mL×2), brine (50 mL), dried over MgSO₄, filtered and evaporated in vacuo. The crude product was purified by silica gel chromatography using ethyl acetate/heptane in different proportions to give a brownish oil. The resulting oil was dissolved in 50 mL of anhydrous ethanol, and 36% hydrochloric acid (1.6 mL) was added. The solvents were evaporated in vacuo, and then co-evaporation with 50 mL of anhydrous ethanol was repeated twice. The obtained concentrated solution (ca. 2 mL) was allowed to cool down in a refrigerator; the precipitated solid was collected by filtration, rinsed with pentane and dried in vacuo overnight. Yield: 505 mg (36%) as a yellowish solid; mp 77-78° C.

### Example 47: (3S)-2,2-dimethyl-9-pentyl-3-[(piperidin-1-yl)carbonyl]-1H,2H,3H,4H,9H-pyrido[3,4-b]indol-2-ium iodide (compound 76)

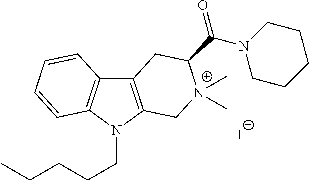 [SMILES: CCCCCn1c2c(c3ccccc31)C[C@@H](C(=O)N1CCCCC1)[N+](C)(C)C2 ; [I-]]

Hydrochloride salt 71 (93 mg, 0.26 mmol) was extracted in ethyl acetate (100 mL) and washed with concentrated aqueous sodium bicarbonate (30 mL×3). The organic phase was washed with water, brine, dried (MgSO₄), filtered, and evaporated in vacuo. The obtained residue was stirred with MeI (100 mL, 1.61 mmol) in 5 mL of methanol for 16 h at room temperature. The organic solvents were removed under reduced pressure, and diethyl ether was added. The precipitated product was collected by filtration and dried in vacuo overnight to give 76 (25 mg, 19% yield) as a yellowish solid; mp 136° C. (with decomposition).

### Example 48: Synthesis of (3R)-2,2-dimethyl-9-pentyl-3-[(piperidin-1-yl)carbonyl]-1H,2H,3H,4H,9H-pyrido[3,4-b]indol-2-ium iodide (compound 77)

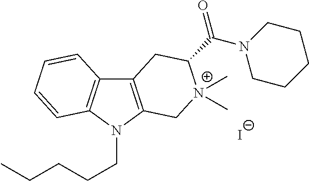 [SMILES: CCCCCn1c2c(c3ccccc31)C[C@H](C(=O)N1CCCCC1)[N+](C)(C)C2 ; [I-]]

Using hydrochloride salt 74 (85 mg, 0.22 mmol) and MeI (260 mL, 4.18 mmol), the title compound was prepared following the procedures described in preparation of compound 76 as a yellowish solid. Yield: 51 mg (46%); mp 207-208° C. (with decomposition).

### Example 49: LC/MS Analyses

LC-MS analyses were performed on a Waters/Micromass LCT, TOF equipped with an Alliance HT Waters 2795 liquid chromatography system and a Waters 2487 dual absorbance detector. The liquid chromatography conditions were as follows: a Phenomenex Gemini-NX 3-um C18 110A 50×4.60 mm column was used and it was eluted with a gradient made up of two solvent mixtures. Solvent A consisted of water and 0.08% TFA. Solvent C consisted of acetonitrile. The gradient was processed as follows:

| Time | A % | B % | C % | D % | Flow (ml/min) |
|---|---|---|---|---|---|
| 0.00 | 90.0 | 0.0 | 10.0 | 0.0 | 0.400 |
| 6.60 | 2.0 | 0.0 | 98.0 | 0.0 | 0.400 |
| 8.00 | 2.0 | 0.0 | 98.0 | 0.0 | 0.400 |
| 8.20 | 90.0 | 0.0 | 10.0 | 0.0 | 0.400 |
| 9.00 | 90.0 | 0.0 | 10.0 | 0.0 | 0.400 |
| 12.00 | 90.0 | 0.0 | 10.0 | 0.0 | 0.400 |

### Example 50: In Vitro Receptor Radioligand Binding Studies

Compounds were screened in a competitive binding experiment using respectively membrane fractions prepared from rat brain homogenate and HEK293 expressing respectively the rat CB1 receptor and hCB2 receptor, at different concentrations, in duplicate. The competition binding experiment for CB1 and CB2 was performed in 96-well plates containing binding buffer (50 mM Tris HCl, 1 mM EDTA, 3 mM MgCl₂, 5 mg/mL fatty acid-free BSA, pH 7.4). The radioligand was [³H]CP55940. The reference was CP55940.

A solution of the compound to be tested is prepared as a 1-mg/mL stock in Standard Binding Buffer or DMSO according to its solubility. A similar stock of a reference compound (positive control) was also prepared. Eleven dilutions (5× assay concentration) of the test and reference compounds are prepared in Standard Binding Buffer by serial dilution.

Radioligand is diluted to five times the assay concentration in Standard Binding Buffer. Aliquots (50 μL) of radioligand are dispensed into the wells of a 96-well plate containing 100 μL of Standard Binding Buffer. Then, duplicate 50-μL aliquots of the test and reference compound dilutions are added. Finally, crude membrane fractions of cells are resuspended in 3 mL of chilled Standard Binding Buffer and homogenized by several passages through a 26 gauge needle, then 50 μL are dispensed into each well.

The 250 μL reactions are incubated at room temperature for 1.5 hours, then harvested by rapid filtration onto Whatman GF/B glass fiber filters pre-soaked with 0.3% polyethyleneimine using a 96-well Brandel harverster. Four rapid 500 μL washes are performed. Filters are placed in 6 mL scintillation tubes and allowed to dry overnight. Bound radioactivity is harvested onto 0.3% polyethyleneimine-treated, 96-well filter mats using a 96-well Filtermate harvester. The filter mats are dried, then scintillant is melted onto the filters and the radioactivity retained on the filters is counted in a Microbeta scintillation counter.

Raw data (dpm) representing total radioligand binding (i.e., specific+non-specific binding) are plotted as a function of the logarithm of the molar concentration of the competitor (i.e., test or reference compound). Non-linear regression of the normalized (i.e., percent radioligand binding compared to that observed in the absence of test or reference compound) raw data is performed in Prism 4.0 (GraphPad Software) using the built-in three parameter logistic model describing ligand competition binding to radioligand-labeled sites:

y=bottom+[(top−bottom)/(1+10×−log IC50)]

where “bottom” equals the residual radioligand binding measured in the presence of 10 μM reference compound (i.e., non-specific binding) and “top” equals the total radioligand binding observed in the absence of competitor. The log IC50 (i.e., the log of the ligand concentration that reduces radioligand binding by 50%) is thus estimated from the data and used to obtain the Ki by applying the Cheng-Prusoff approximation:

Ki=IC50/(1+[ligand]/KD)

where [ligand] equals the assay radioligand concentration and KD equals the affinity constant of the radioligand for the target receptor.

### Example 51: GTPγ[³⁵S] Functional Assays

Functional activity was evaluated using GTPγ[³⁵S] assay in CHO membrane extracts expressing recombinant hCB1 (human CB1) receptors or hCB2 (human CB2) receptors. The assay relies on the binding of GTPγ[³⁵S], a radiolabeled non-hydrolyzable GTP analogue, to the G protein upon binding of an agonist of the G-protein-coupled receptor. In this system, agonists stimulate GTPγ[³⁵S] binding whereas neutral antagonist have no effect and inverse agonists decrease GTPγ[³⁵S] basal binding.

Compounds were solubilized in 100% DMSO at a concentration of 10 mM within 4 hours of the first testing session (master solution). A predilution for the dose response curve was performed in 100% DMSO and then diluted 100 fold in assay buffer at a concentration 2 fold higher than the concentration to be tested. Compounds were tested for agonist and antagonist activities in duplicate with CP55,940 (Tocris, Bioscience, Ellisville, Mich., USA) as reference agonist. For GIPγS membranes were mixed with GDP diluted in assay buffer to give 30 μM solution (volume:volume) and incubated for at least 15 min. on ice. In parallel, GIPγ[³⁵S] (GE Healthcare, Catalogue number SJ1308) were mixed with the beads (PVT-WGA (GE Healthcare, RPNQ001)), diluted in assay buffer at 50 mg/mL (0.5 mg/10 μL) (volume:volume) just before starting the reaction. The following reagents were successively added in the wells of an Optiplate (Perkin Elmer): 50 μL of ligand, 20 μL of the membranes:GDP mix, 10 μL of assay buffer for agonist testing, and 20 μL of the GTPγ[³⁵S]:beads mix. The plates were covered with a topseal, shacked on an orbital shaker for 2 min., and then incubated for 1 hour at room temperature. The plates were then centrifuged for 10 min. at 2000 rpm and counted for 1 min./well with a PerkinElmer TopCount reader. Assay reproducibility was monitored by the use of reference compound CP 55,940. For replicate determinations, the maximum variability tolerated in the test was of ±20% around the average of the replicates. Efficacies (Eₘₐₓ) for CB1 or CB2 are expressed as a percentage relative to the efficacy of CP 55,940.

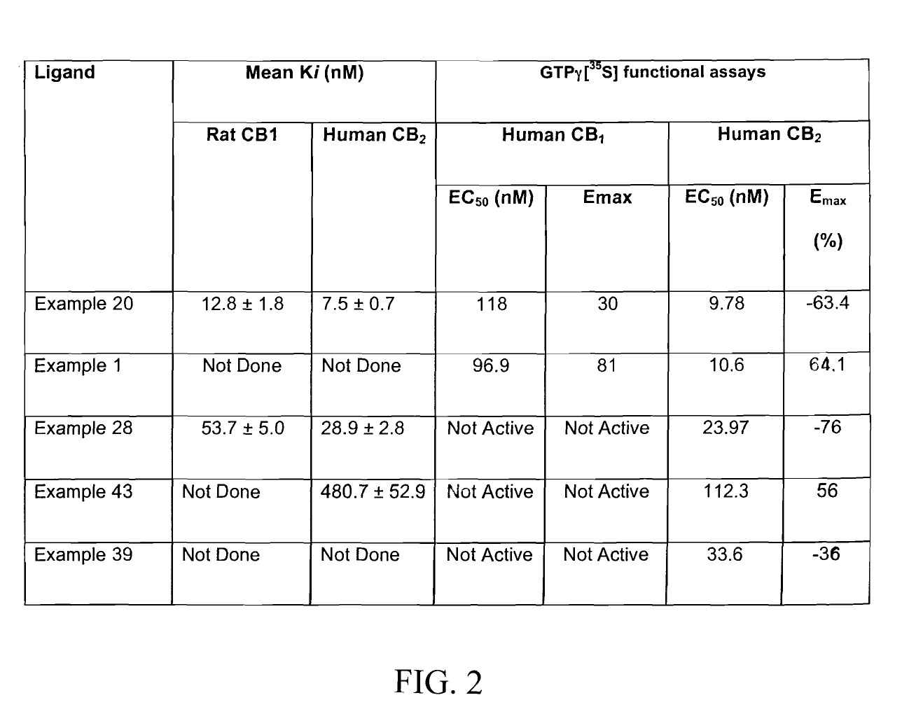

Using the above-mentioned assays, the compounds of the invention were found to be active towards CB1 and CB2 receptors (FIG. 2).

### All Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are within the claims.

What is claimed is:

1. A compound represented by Formula I: 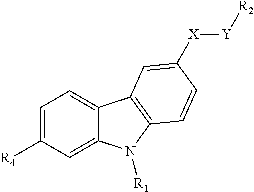
or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, a hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, alkyl, cycloalkyl, aralkyl, alkenyl, alkynyl, a halogen, alkoxy, and hydroxyl.

2. A compound represented by Formula II: 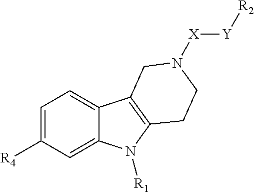
or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently selected from the group consisting of hydrogen, alkyl, acyl, heteroalkyl, aryl, cycloalkyl, heteroaryl, and heterocycloalkyl, any carbon atom of which may be optionally substituted; and
R⁸ is selected from the group consisting of hydrogen, aryl, alkyl, cycloalkyl, aralkyl, alkenyl, alkynyl, a halogen, alkoxy, and hydroxyl.

3. A compound represented by Formula III: 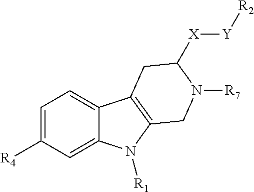
or a salt, ester or prodrug thereof, wherein
X is selected from the group consisting of C═O, C═S, S═O and SO2;
Y is selected from the group consisting of O, N—R³, and R³—C—R⁸;
R¹, R², and R³ vary independently and are selected from the group consisting of alkoxy, alkylcarbonyl, polyether radical, heteroaryl, heterocycloalkyl, aryl, alkyl, cycloalkyl, aralkyl, alkenyl, and alkynyl;
R⁴ is selected from the group consisting of hydrogen, aryl, alkyl, cycloalkyl, aralkyl, alkenyl, alkynyl, heteroaryl, a halogen, alkoxy, heterocycloalkyl, polyether radical, hydroxyl, and R⁶—N—R⁵;
R⁵ and R⁶ vary independently and are selected from the group consisting of hydrogen, alkyl, acyl, heteroalkyl, aryl, cycloalkyl, heteroaryl, and heterocycloalkyl, any carbon atom of which may be optionally substituted;
R⁷ is selected from the group consisting of, polyether radical, alkylcarbonyl, heteroaryl, heterocycloalkyl, aryl, alkyl, cycloalkyl, aralkyl, alkenyl, and alkynyl; and
R⁸ is selected from the group consisting of hydrogen, aryl, alkyl, cycloalkyl, aralkyl, alkenyl, alkynyl, a halogen, alkoxy, and hydroxyl.

4. The compound of claim 1, wherein said compound is selected from the group consisting of N-[2-(4-chlorophenyl)ethyl]-9-pentyl-9H-carbazole-3-carboxamide; 9-(cyclohexylmethyl)-2-methoxy-6-[(piperidin-1-yl)carbonyl]-9H-carbazole; 2-methoxy-9-pentyl-6-[(piperidin-1-yl)carbothioyl]-9H-carbazole; 9-pentyl-3-[(piperidin-1-yl)carbothioyl]-9H-carbazole; 9-pentyl-6-[(piperidin-1-yl)carbonyl]-9H-carbazol-2-ol; 2-[(ethylsulfanyl)methoxy]-9-pentyl-6-[(piperidin-1-yl)carbonyl]-9H-carbazole; 2-methoxy-6-[(morpholin-4-yl)carbonyl]-9-pentyl-9H-carbazole; 3-[(morpholin-4-yl)carbonyl]-9-pentyl-9H-carbazole; 2-methoxy-6-[(4-methylpiperazin-1-yl)carbonyl]-9-pentyl-9H-carbazole; 3-[(4-methylpiperazin-1-yl)carbonyl]-9-pentyl-9H-carbazole; 7-methoxy-9-pentyl-N-(piperidin-1-yl)-9H-carbazole-3-carboxamide; 9-pentyl-N-(piperidin-1-yl)-9H-carbazole-3-carboxamide; N,N-diethyl-7-methoxy-9-pentyl-9H-carbazole-3-carboxamide; N,N-diethyl-9-pentyl-9H-carbazole-3-carboxamide; N-(adamantan-1-yl)-7-methoxy-9-pentyl-9H-carbazole-3-carboxamide; N-(adamantan-1-yl)-9-pentyl-9H-carbazole-3-carboxamide; 2-(methylsulfanyl)-9-pentyl-6-[(piperidin-1-yl)carbonyl]-9H-carbazole; 2-methanesulfonyl-9-pentyl-6-[(piperidin-1-yl)carbonyl]-9H-carbazole; N-[2-(4-chlorophenyl)ethyl]-7-methoxy-9-pentyl-9H-carbazole-3-carboxamide; 4-[(7-methoxy-9-pentyl-9H-carbazol-3-yl)carbonyl]-1,1-dimethylpiperazin-1-ium iodide; 1,1-dimethyl-4-[(9-pentyl-9H-carbazol-3-yl)carbonyl]piperazin-1-ium iodide; 4-[(9-pentyl-9H-carbazol-3-yl)carbonyl]-1λ⁶,4-thiomorpholine-1,1-dione; dimethyl(3-{3-[(piperidin-1-yl)carbonyl]-9H-carbazol-9-yl}propyl)amine; 9-(3-methoxypropyl)-3-[(piperidin-1-yl)carbonyl]-9H-carbazole; methyl 4-{3-[(piperidin-1-yl)carbonyl]-9H-carbazol-9-yl}butanoate; 3-benzoyl-9-pentyl-9H-carbazole; 9-(oxan-4-ylmethyl)-3-[(piperidin-1-yl)carbonyl]-9H-carbazole; 3-[(piperidin-1-yl)carbonyl]-9-(pyridin-4-ylmethyl)-9H-carbazole; 3-[(piperidin-1-yl)carbonyl]-9-(pyridin-3-ylmethyl)-9H-carbazole; 9-ethyl-3-[(4-methylnaphthalen-1-yl)carbonyl]-9H-carbazole; 2-methoxy-9-pentyl-6-[(piperidin-1-yl)carbonyl]-9H-carbazole; 9-pentyl-3-[(piperidin-1-yl)carbonyl]-9H-carbazole; 3-[(piperidin-1-yl)carbonyl]-9-(pyridin-2-ylmethyl)-9H-carbazole; methyl 9-(cyclohexylmethyl)-7-methoxy-9H-carbazole-3-carboxylate; ethyl 9-pentyl-9H-pyrido[3,4-b]indole-3-carboxylate; N-(2,2-dimethylpropyl)-9-pentyl-9H-pyrido[3,4-b]indole-3-carboxamide; 1-({9-pentyl-9H-pyrido[3,4-b]indol-3-yl}carbonyl)piperidine; 9-pentyl-N-(piperidin-1-yl)-9H-pyrido[3,4-b]indole-3-carboxamide, 2-methoxy-9-pentyl-6-(piperidin-1-ylmethyl)-9H-carbazole; methyl 9-[3-(dimethylamino)propyl]-7-methoxy-9H-carbazole-3-carboxylate; and 2-(dimethylamino)ethyl 9-pentyl-9H-carbazole-3-carboxylate.

5. The compound of claim 2, wherein said compound is selected from the group consisting of 2-benzoyl-7-methoxy-5-pentyl-1H,2H,3H,4H,5H-pyrido[4,3-b]indole; 5-{7-methoxy-5-pentyl-1H,2H,3H,4H,5H-pyrido[4,3-b]indole-2-sulfonyl}-N,N-dimethylnaphthalen-1-amine; and 5-ethyl-7-methoxy-2-[(4-methylnaphthalen-1-yl)carbonyl]-1H,2H,3H,4H,5H-pyrido[4,3-b]indole.

6. The compound of claim 3, wherein said compound is selected from the group consisting of 1-{[(3R)-9-pentyl-1H,2H,3H,4H,9H-pyrido[3,4-b]indol-3-yl]carbonyl}piperidine hydrochloride; 1-{[(3S)-9-pentyl-1H,2H,3H,4H,9H-pyrido[3,4-b]indol-3-yl]carbonyl}piperidine hydrochloride; (3R)-2,2-dimethyl-9-pentyl-3-[(piperidin-1-yl)carbonyl]-1H,2H,3H,4H,9H-pyrido[3,4-b]indol-2-ium iodide; (3S)-2,2-dimethyl-9-pentyl-3-[(piperidin-1-yl)carbonyl]-1H,2H,3H,4H,9H-pyrido[3,4-b]indol-2-ium iodide; and 1-{[(3R)-2,9-dipentyl-1H,2H,3H,4H,9H-pyrido[3,4-b]indol-3-yl]carbonyl}piperidine hydrochloride.

7. The compound of claim 1, further comprising at least one imaging agent.
